(19) United States
(12) Patent Application Publication
(10) Pub. No.: US 2021/0396737 A1
(43) Pub. Date: Dec. 23, 2021

(54) NANOPLEXED POLY(I:C) FORMULATIONS AND USES THEREOF

(71) Applicant: HIGHLIGHT THERAPEUTICS, S.L., Paterna, Valencia (ES)

(72) Inventors: MARISOL ORTIZ QUINTERO, Paterna, Valencia (ES); DOMINQUE TERSAGO, Paterna, Valencia (ES); JULIO M RÍOS DE LA ROSA, Paterna, Valencia (ES); PEDRO P LOPEZ-CASAS, Paterna, Valencia (ES)

(21) Appl. No.: 17/295,262

(22) PCT Filed: Nov. 21, 2019
(86) PCT No.: PCT/EP2019/082161

§ 371 Date: May 19, 2021

(30) Foreign Application Priority Data
Nov. 21, 2018 (EP) 18207579.6

(51) Int. Cl. G01N 33/50; A61K 31/713; A61K 47/69; A61K 45/06; C07K 16/28; A61K 9/08

(52) U.S. Cl. CPC G01N 33/5011; A61K 31/713; A61K 47/6929; B82Y 5/00; C07K 16/2818; C07K 16/2827; A61K 9/08; A61K 45/06

(57) ABSTRACT

Immune cells and cancer cells that can internalize nanoplexed poly(I:C) formulations more efficiently, and are therefore more effectively modified by such formulations, are presently characterized both in vitro and in vivo using novel labeled nanoplexed poly(I:C) formulations. The internalizing of the disclosed formulations by the cells allows the defining of specific medical indications and/or subject patient populations, in particular in subjects presenting cancer, in addition to the identification of preferred routes and regimens for beneficially administering a nanoplexed poly(I:C) formulation, alone or in combination with other drugs, to achieve desired therapeutic outcomes for such patients.

### CROSS-REFERENCE TO RELATED APPLICATIONS

This application is a national phase entry under 35 U.S.C. § 371 of International Patent Application PCT/EP2019/082161, filed Nov. 21, 2019, published as International Patent Publication WO 2020/104628 on May 28, 2020, which claims the benefit of European Patent Application 18207579.6, filed on Nov. 21, 2018, the contents of all are hereby incorporated by reference.

### FIELD OF THE INVENTION

The present invention relates an improved detection, evaluation, and clinical use of pharmaceutical formulations comprising particles formed by a combination of polyribonucleotides and polymers.

### BACKGROUND OF THE INVENTION

The use of synthetic analogs of double-stranded RNA (dsRNA) capable of mimicking viral dsRNA has been investigated in recent years, in particular their role in specifically activating the immune system against tumors, with the aim of inhibiting cancer cell growth and inducing cancer cell apoptosis. In particular, double-stranded polyinosinic-polycytidylic acid (known as poly(I:C) or plC) has been characterized as a type of dsRNA offering various therapeutic effects against several types of cancers and their metastasis, effects achieved dependently or independently from immune system activation, natural killer- and/or dendritic cell-mediated activities, and/or changes in tumor gene expression and the cellular microenvironment.

Over the last few years, there has been significant progress reported in formulating poly(I:C) molecules with immunomodulatory and/or therapeutic properties. Methods of preparing and formulating poly(I:C) molecules within polymer-based microparticles with or without targeting moieties, carrier polymers, and chemical linkers have been disclosed for various dosages, administration regimes, and medical indications.

Disadvantages are associated with the clinical development of poly(I:C) molecules as a drug and its compliance with demanding regulatory requirements could be overcome by producing structurally complex anticancer complexes comprising poly(I:C) molecules, together with drug delivery systems for cancer therapy, that are often based on cationic polymers such as chitosan, polyethyleneimine (PEI), or poly-L-lysine. These polymeric systems are also known as Polyplex systems, or more specifically, nanoplex systems, the latter corresponding to a complex formed by a drug nanoparticle with an oppositely charged polyelectrolyte (Kadam, R N et al., 2015).

Among Polyplex options described for products based on polyriboinosinic: polyribocytidylic acid (Patel M C et al., 2014), PEI represents a polyalkyleneimine of particular interest being a cationic polymer that can be modified and adapted at various levels. This approach involving the preparation and the administration of poly(I:C) molecules associated to PEI, and acting as TLR3, RIG-I, and/or MDA5 agonists, has been exemplified in literature by an agent known as BO-110 (Tormo D et al., 2009; WO2011/003883). The initial approach for manufacturing the BO-110 product has been improved by a means of a process providing nanoplex particles featuring highly controlled, uniform size, high concentration for poly(I:C) molecules, and beneficial biological activities such as strong, tumor-targeting therapeutic effects. This manufacturing process and the resulting pharmaceutical compositions (referred to generally as BO-11X, exemplified by one formulation known as BO-112) have been described in PCT/EP2016/078078 and PCT/EP2017/079688, wherein BO-112 biological and therapeutic activities were tested in a variety of cell-based systems, animal models, and in clinical studies, in particular by intratumoral injection. In a Phase 1 study, BO-112 was found to be safe as a monotherapy or as a combination therapy with anti-PD1 blockade in patients presenting solid tumors (Marquez Rodas I et al., 2018).

However, to date the pre-clinical and clinical characterization of the BO-11X formulations still requires further characterization of underlying mechanism of action, cellular localization and biodistribution after administration, and interaction with cells in human body (mainly by cell internalization), that would establish a viable medical use for BO-11X as a drug, for example, a cancer therapeutic agent, not only for specific conditions, presently applied treatment protocols, and/or dosage regimens, but also potentially therapeutically useful for a specific sub-population of patients. Indeed, aside from improving general clinical evaluation and use of BO-11X together with patient compliance, it is important to characterize cell type-specific, BO-11X interactions, localization within or nearby tumors and other tissues, and any other physiological effects in parallel to a candidate patient's molecular and biological profile for using BO-11X formulations with well-defined safety margins and therapeutic effects among cancer patients having comparable clinical status, but differing responses to cancer treatments, in particular to standard-of-care protocols (such as radiotherapy), antibodies targeting cancer antigens, or cancer immunotherapies.

### SUMMARY OF THE INVENTION

The present invention relates to the characterization of cell-specific BO-11X interactions, and corresponding therapeutic effects both in vitro and in vivo, in particular in a clinical setting. In one embodiment, the present disclosure relates to novel nanoplexed formulations of one or more particles formed from PEI and Poly(I:C) molecules acting as a therapeutic composition that, in addition of acting as agonist of Toll-Like receptor 3 (TLR3) and/or a cytoplasmic double stranded RNA (dsRNA) sensor (such as RIG-I or MDA5), are labeled within PEI and/or Poly(I:C) molecules (referred to as BO-11XL formulations), preferably with a fluorophore.

The present invention involves the use of BO-11X formulations comprising efficiently labeled nanoplex particles, that allow identifying cells within tissues or organs (such as lymph nodes, skin, or internal organs like liver or lungs) that preferably interact with similar types of complexes characterizing BO-11X formulations once the formulation is administered, in particular intratumorally or by other type of injection. The disclosed BO-11XL formulations allow identifying and tracking the localization and/or the changes in number and biological features of immune cells and cancer cells that preferentially interact with the disclosed nanoparticles. BO-11XL and BO-11X formulations exert distinct biological effects of therapeutic interest on such cells (such as modifying cell markers, gene expression, or viability) and/or modifying the activities of such cells in human body by alternative biological mechanisms, in particular within tumors or lymph nodes, preferably wherein the cells are derived from a mammalian subject, including a human subject.

These findings, when analysed and compared to clinical read-outs or therapeutic effects observed in cancer patients treated (or who are candidates for treatment) with a BO-11X formulation, and thus present cells that interact with the nanoplexed formulations, advantageously allow defining novel clinical uses of BO-11X formulations, alone or in combination as a first therapeutic composition with a second therapeutic agent including a conventional cancer treatment (such as standard-of-care and/or cancer immunotherapies), either during or prior to the treatment directed to specific types of cancer or populations of patients affected by cancer, including those that may particularly benefit by more appropriate dosages, regimens, or methods of administering drugs such as an antibody directed to a cancer antigen or an immune checkpoint. Such methods for determining drug regimens for the treatment of cancer comprise detecting effects of drug formulations comprising nanoplex particles within biological samples either in vitro or ex vivo (using cell lines, tumor biopsies, or blood samples obtained from cancer patients).

In the latter case, subjects identified as candidates for BO-11X treatment (as a first line or later line of treatment) may be selected for such treatment, or identified to be preferably treated with a BO-11X formulation, since presenting specific parameters (e.g. biomarkers) and/or not responding to conventional standard-of-care cancer therapies or cancer therapies that targets to specific cancer antigens, immune checkpoints, cells, or biological pathways, and involving cancer therapies of a different chemical nature, such as small molecules, peptides, antibodies, cell-based products, or nucleic acids.

Indeed, the above-described validation process of a BO-11X formulation may additionally lead to characterizing BO-11X formulations in which alternative size ranges of particles, PEI molecules, and/or Poly(I:C) molecules are tested using different in vitro, in vivo, and/or ex vivo models to identify those providing the most therapeutically and/or pharmaceutically relevant effect on cancer cells and/or immune cells, thus providing useful BO-11X formulations in addition to the BO-112 formulation.

The present invention further relates to optimized medical uses of BO-11X formulations, such as improved methods and regimens for cancer treatments with respect to specific protocols and/or combinations, in particular those including the administration of an antibody as a second therapeutic agent directed to a cancer antigen (such as CD20 or Her2) and/or an immune checkpoint (such as PD-1 or PD-L1), methods for monitoring clinical responses to BO-11X treatment and adapting such treatment, methods for evaluating potential clinical responses of cancer patients prior to BO-11X treatment, or methods for treating cancer or for improving pharmaceutical compositions that comprise a BO-11X formulations in specific indications and/or for specific cancer patients. These methods may also comprise the comparison with standardized assessment criteria for clinical response that are determined for such patients, such those defined under RECIST, irRC, and/or PERCIST standardized criteria that would allow determining optimized drug regimens by using appropriate biological samples obtained from said patients (preferably biopsies or blood samples) and analytical technologies commonly used (such as flow cytometry, in vivo imaging, or ELISA).

Further embodiments relate to novel, improved BO-11X formulations, uses and methods according to the present invention are disclosed herein in the following detailed description and in the Examples below.

### BRIEF DESCRIPTION OF THE DRAWINGS

FIG. 1: Comparative biophysical features, cell uptake and effects of standard BO-112 (BO-112) or of a fluorescently labeled BO-11X formulation (BO-11XL). Three BO-11X formulations, unlabeled BO-112, BO-11XL comprising Poly(I:C) molecules labeled with Rhodamine (BO-11XL pIC:Rho) and BO-11XL comprising PEI molecules labeled with Cy5 (BO-11XL PEI:Cy5) were compared by Dynamic Light Scattering (DLS) for a series of features including size distribution that appear highly similar (A). The BO-11XL(pIC:Rho) formulation was further tested using two murine cell lines, a OVA-transfected, B16-derived melanoma cell line (B16-OVA; B) or DC2.4 dendritic cell line (DC2.4; C), to evaluate the uptake of BO-11XL (1 μg/mL) via flow cytometry on live cells by following the median fluorescence intensity (MFI) and percentage of positive cells over time (t=5, 10, 30 and 60 min). Untreated cells were used as a threshold to calculate the percentage of positive cells at each time point. The effect on cell viability was measured via MTS assay after 48 hours incubation with 1 μg/mL BO-112 in cell culture media, using untreated cells as negative control (see right graph in panels B and C). When this cell viability analysis is pursued up to 72 hours, the BO-112 treatment results in cell proliferation, which is indicative of dendritic cell activation (D). Data are presented as average ±SD (n=3).

FIG. 2: Evaluating the uptake of a BO-11XL formulation in different cell culture models. Cell cultures of the indicated cell lines are incubated in media where BO-11XL (pIC:Rho) is added. Sample of cells are isolated at different time points over 72 hours and analyzed by flow cytometry for calculating the median fluorescence intensity (MFI) due to the update of the labeled particles and the percentage of positive, labeled cells in each sample (B). The flow cytometry data were confirmed by immune fluorescence in confocal microscopy of fixed MC38 (colorectal cancer) and DC2.4 (dendritic cell line) identifying typical red, Rhodamine fluorescent signals in cell cytoplasm after counter-staining cell nuclei in blue with DAPI (4′,6-diamidino-2-phenylindole), a DNA-specific fluorescent probe.

FIG. 3: Detecting lymphatic uptake of BO-11XL ex vivo following intratumoral injection in an animal model. The model is established by injecting MC38 (colorectal cancer cells) in right flank of each mouse, nearby an auxiliary lymph node (A; the position in mouse body and names of the relevant lymph nodes are indicated). The in vivo fluorescence of BO-11XL(pIC:Rho) that is injected in MC38-originated tumor is detected after 24-48 hours within the tumor itself (used as reference signal) and the indicated lymph nodes (B). In parallel, specific immune cells such as CD45+cells (covering B cells, NK cells, CD4-positive and CD8-positive cells) can be isolated from each lymph nodes to evaluate ex vivo the number of labeled cells (left panel) and fluorescence intensity (right panel) by flow cytometry in animals that are sacrificed at the indicated time points (C, including details for proximal lymph nodes only).

FIG. 4: Detecting lymphatic uptake of BO-11XL in vivo following intratumoral injection in an animal model. Using the same mouse model described in the previous figures, the fluorescence is detected within mouse at 1 hour, 6 hours, 24 hours, and 48 hours post-intratumoral (i.t.) injection of BO-11XL(pIC:Rho) either in the lateral position (A) or, after surgery, in the supine, open position (B), in the latter case comparing the signal that is detected in tumor and the ipsilateral lymph nodes, with a lower but still measurable signal also detected in contralateral ones. The signal that is measured at each time point is shown in the graph together with a representative image of an injected mouse in either position.

FIG. 5: Alternative methods and drug regimens for BO-11X administration in combination with a cancer immunotherapy such as the intra-venous injection of anti-PD1 (e.g. pembrolizumab) or anti-PD-L1 antibodies (anti-PD1/PD-L1; 0.1-1.0 mg/dose, e.g. 200 mg/dose). The administration of a BO-11X formulation such as a BO-112 formulation by intratumoral injection (BO-112 IT; 0,4-2,4 mg/dose) may be started on a schedule either before the beginning of a standard anti-PD1/PD-L1 protocol (A and B) or concomitantly with a standard anti-PD1/PD-L1 protocol (C and D). The standard anti-PD1/PD-L1 protocol may be performed by administering anti-PD1/PD-L1 either every 3 weeks (q3w; panels A and C) or every 2 weeks (q2w; panels B and D). The administration of the BO-112 formulation by intratumoral injection may be performed every week (or day) that anti-PD1/PD-L1 is also administered in combination, or injection may not be performed at specific weeks (e.g. at Week 10, 18, and 22 in the exemplary drug regimen shown in panel B or at Week 7, 11, 19, and 23 in the exemplary drug regimen shown in panel D). Otherwise, the administration of BO-112 formulation by intratumoral injection may be performed during a week in which anti-PD1/PD-L1 is not administered (e.g. at Week 2 in the exemplary drug regimen shown in panels C and D). The specific drug regimen identified by the box in each panel represents a maintenance drug regimen that can be performed for a number of cycle that depends from the patient's clinical status and responses (for example, a cycle of 12 weeks that is repeated 2, 3, or 4 times; see Weeks 12-23 in panels A and B, Week 13-24 in panels C and D). The drug regimens exemplified in FIG. 5 may involve the injection of the same lesion (if still present) or another lesion if the original lesion is no longer present (e.g. by systemic immune activation), or as evaluated by clinician in view of other clinical parameters such as the clinical response defined according to an analysis of biomarkers, cancer antigens, immune cells, or clinical criteria (e.g. tumor burden, stage of the tumor, amount of metastasis, and/or tumor recurrence).

FIG. 6: Effect of intratumoral injection of BO-112 formulation in a subject affected by renal cell carcinoma. The decrease in size of injected and non-injected tumor lesions (A) is associated with combined variations in immune cell populations (B). The comparison of clinically relevant effects such as the size of injected (or not) tumor lesions with biological features, such as changes in amount, distribution, or markers in immune cells or cancer cells may allow defining the efficacy and alternative drug regimens applicable to BO-11X-based treatment of cancer, during or before the actual treatment.

FIG. 7: Volcano plot representing the change in gene expression when using Nanostring to compare tumor samples coming from patients that had as best response PR (Partial Response) or SD (stable disease), with samples from patients whose best response was disease progress (no benefit) after treatment with BO-112 and an anti-PD-1 antibody. Gene expression was assessed in tumor samples collected after three doses of BO-112 treatment, using Nanostring nCounter technology. Evaluation of BO-112 intratumoral injection on gene expression was focused on categories of genes known to be associated with various therapeutically relevant signaling pathways or immune responses, such as those related to interferon gamma or to Cytotoxic T lymphocytes (CTL)-mediated cytotoxicity. In addition to those indicated in the Volcano plot, other human genes that were identified with statistically significant (P<0.005) up-regulated expression include the genes S100A8 (S100-A8, Calgranulin-A), RRAD (GTP-binding protein RAD), CXCL13 (C-X-C motif chemokine 13), SPINK5 (Serine protease inhibitor Kazal-type 5), CCL21 (C-C motif chemokine 21), GNLY (Granulysin), CXCL9 (C-X-C motif chemokine 9), SLAMF1 (Signaling lymphocytic activation molecule), TNFRSF17 (Tumor necrosis factor receptor superfamily member 17, CD269), and CCL19 (C-C motif chemokine 19). Similarly, other human genes that were identified with statistically significant (P<0.005) down-regulated expression include CD36 (Platelet glycoprotein 4) and ApoE (Apolipoprotein E).

FIG. 8: Alternative approaches for evaluating and using a BO-11X formulation such as a BO-112 formulation in cancer patients. (A) Flowchart summarizing medically relevant readouts to evaluate cancer patients as being eligible for the effective administration of a BO-11X formulation such as BO-112 as a monotherapy, as first line or subsequent line of treatment. Prior to BO-11X administration, the eligibility of a candidate cancer patient can be determined (see box at top right side) by measuring the presence or absence of specific (sub)populations of cancer cells or immune cells as well as mutations, down-regulation, or other changes in the expression and/or activity level of genes and pathways involved in antigen presentation (such as B2M or NLRC5) or the presence and/or activity of cross-presenting Dendritic cells (such as CD141-positive DCs). These features may also be relevant for anticipating the preferable use of a BO-11X formulation instead of other alternative innate immune agonists (such as TLR9 agonists, TLR6 agonist, TLR8 agonists, TLR4 agonists, or STING agonists) in these candidate patients. Follow-up of medical conditions can be performed by means of, for example, physical examination and imaging techniques for identifying and measuring tumor burden, immune-related response criteria in cancer treatment, using standardized assessment criteria (such as those defined as WHO criteria, RECIST, irRC, or PERCIST), and/or monitoring cancer-specific biomarkers in blood to evaluate response to treatment, or recurrence. A series of sub-criteria can be associated to each of the three main evaluation criteria (i.e. boxes at the bottom of the flowchart), involving the evaluation of specific parameters or biomarkers in tumor tissue obtained via biopsy and/or in blood samples, including the modification in the expression of and/or response to cytokines (such as interferons or interleukins, individually or in specific combinations) or circulating/infiltrating immune cells that are defined by combinations of markers. (B) Flowchart summarizing the therapeutic opportunities that are available following (but possibly started even before or concurrently with) the administration of a BO-11X formulation such as intratumoral injections of a BO-112 formulation in combination with another anti-cancer drug (in particular checkpoint inhibitors, or CPI). These approaches (see box at top right side) may compare pharmacological or biological features relevant for the therapeutic efficacy of the drug to be administered in combination with a BO-11X formulation (such as scoring the positivity for relevant cell surface receptors in tumor biopsies, e.g. changes in the percentage of cells expressing PD-L1 and/or in PD-L1 expression level in cells), in addition to the read-outs as defined in the previous flowchart (such as those coming from the analysis of gene expression, protein expression, mutational burden status, or T cell clonality in specific cells or tissues before and after BO-112 administration). This combined analysis provides a means for determining eligibility of the cancer patient for a combined treatment regimen, for predicting the effectiveness of the combined treatment, and/or identifying the molecular signatures related to the combined treatment that may indicate (see boxes in the bottom of the flowchart) a continuation with the same or different drug regimen of BO-11X and/or combined cancer drug treatment, or a different type of medical follow-up, including the possibility to use other additional treatments or interventions (e.g., radiotherapy, chemotherapy, anti-PD-1 or another immunomodulating therapy, cancer vaccination, adoptive cell transfer, etc.) against which the tumor in the patient was identified as being poorly responding, resistant or insensitive prior to the administration of BO-11X formulation in combination with such other cancer drug.

### DETAILED DESCRIPTION OF THE INVENTION

The present invention relates to in vivo and in vitro cell-specific interactions and effects provided by a novel BO-11X formulation, which can be therapeutically exploited for evaluating a means for improving the detection of effects and clinical uses of a BO-11X formulation as a monotherapy, or as part of a combination drug regimen and/or other therapeutic formulations useful in a clinical setting, in particular for cancer treatment or vaccination applications. These improvements provided herein have been observed following the analysis of biological samples obtained from patients treated with a BO-11X formulation, such as a BO-112 formulation, as well as by the preparation of BO-11X formulations in which the BO-11X nanoplex particles are efficiently labeled (defined herein as BO-11XL formulations). BO-11XL formulations can be tested in cell-based or animal models, in particular with respect to cell viability and/or cell type distribution, such that the therapeutically relevant properties of BO-11X formulations can be suitably evaluated, or predicted, as needed, by modifying the Poly(I:C) and/or PEI features so that a more desirable cancer-specific and/or immune-specific effects can be obtained using different BO-11X formulations. Such biological effects of BO-11X formulations can be detected and compared either in vitro in panels of cell lines of different origin (so that cell type-specific effects can be determined), or in vivo/ex vivo following administration of the BO-11XL formulations in animal models (defining the pattern of biological responses).

The BO-11XL formulations described herein present biological and biophysical features comparable with features of earlier BO-11X formulations, such as BO-112 formulations, and allow both qualitative and quantitative evaluation of cells, in particular immune cells and cancer cells, that preferentially interact with the nanoplex particles present in the BO-11XL formulations. The analysis of data obtained by exposing cell preparations, tissues, organs, animals, organoids, human or clinical samples (such as biopsies, blood or plasma preparations) or other biological samples or extracts indicative of a pathology (such as cancer or a viral infection) to BO-11XL formulations permits defining which distinct biological targets and effects are a direct result of, or associated with, the activities that the disclosed BO-11X formulations exert on such cells (such as apoptosis, autophagy, activation, proliferation, or other) or such cells later exert in the human body (within the tumor, in lymph nodes, in blood, etc.). The biological effects of BO-11XL formulations may be detected and defined using a variety of physiological criteria (such as changes in the combined secretion of chemokines, cytokines, interferons, etc., or in the expression of specific cell surface receptor or transcription factors). Such findings, when compared to clinical or therapeutic effects in cancer models or patients that are treated (or are a potential candidate of treatment) with a BO-11X formulation in parallel or in comparable situations, advantageously permit the identification of novel clinical uses, methods of treatment and drug regimens that may benefit from treatment by BO-11X formulations, alone or in combination with other drugs, including anti-cancer drugs (such as standard-of-care, antibodies against cancer antigens, or cancer immunotherapies), or antigens (human or non-human ones, such as in vaccination), either during the course of treatment or prior to the treatment.

In particular, the present invention allows defining methods of treatment and medical regimens that may benefit from the administration of a BO-11X formulation, such as a BO-112 formulation, suitable for a given pathology (i.e. a specific type of cancer according its pathophysiology, metastatic properties, location, stage, etc., or a specific vaccination approach), populations of candidate patients (i.e. as defined by prior treatments, ongoing standard-of-care or other treatment, immune profile, altered copy number, type of infection, genetic mutations or activation of relevant genes), and/or combination with other drugs (such as checkpoint inhibitors, antibodies targeting cancer antigens, cancer vaccines, non-human antigens, or adoptive cell therapies). In addition, the present invention also provides means for defining which patients treated (or are a candidate for treatment) with a specific type of therapeutic compound, could be preferably treated with a BO-11X formulation, since the mechanism by which such therapeutic compound would be therapeutically active is ineffective or otherwise inefficient. The comparison of effects exerted by a BO-11XL formulation and/or of a BO-11X formulation with such other therapeutic compounds in clinically relevant models advantageously permits the identification of present cells, tumors, and/or an overall pathological or immunological status of a patient, indicating that such patient would preferentially and positively respond to a BO-11X-based treatment Such a comparison may also be beneficial in determining which drug regimen(s) (i.e. monotherapy or a drug combination) may be more adaptable in a given patient or sub-populations of patients, and/or disease stage or type (as those generally defined in cancer or infections).

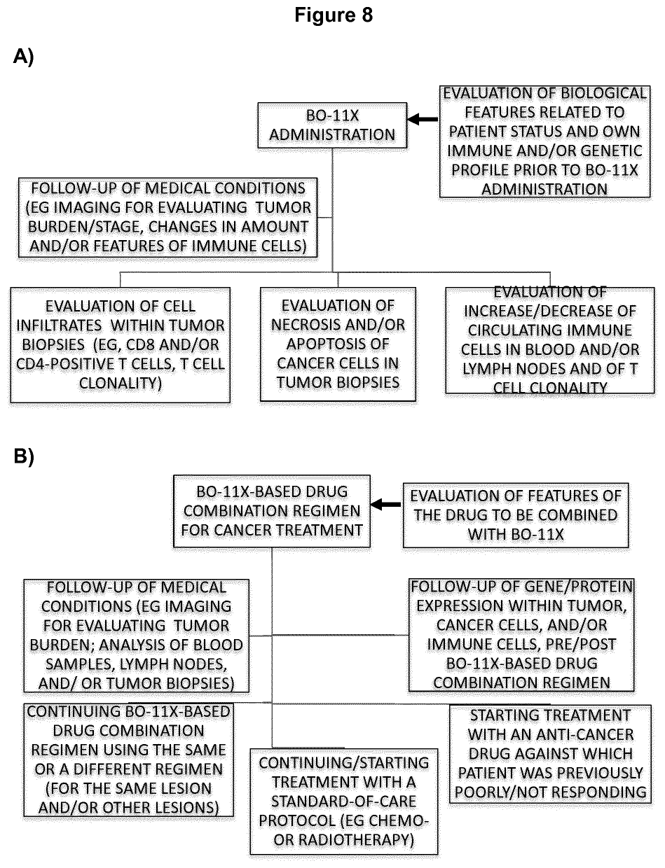

These alternative, novel therapeutic drug regimens may involve the administration of a BO-11X formulation by injection into the blood stream (so that different tissues may be exposed to the formulation) or at specific physiological locations, such as directly in an organ, skin, and/or pathological altered tissues, such as cancer lesions. In this latter case, administration of a BO-11X formulation may be performed in the same lesion (if still present) or another lesion if original lesion is no longer present (through systemic immune activation or other mechanism) or as evaluated by clinicians in view of other clinical parameters such as a suitable clinical response defined by the analysis of biomarkers, gene expression signatures, cancer antigens, immune cells, or clinical criteria (e.g., tumor burden, stage of the tumor, amount of metastasis, and/or tumor recurrence). Additional details on how the drug regimen and course of treatment may be adapted to either monotherapy or drug combination treatments involving a BO-11X formulation are shown in FIG. 8. Moreover, the relevant institutions responsible for the evaluation and/or control of clinical therapeutics (such as FDA, EMA, WHO and ICLIO) provide a series of criteria for evaluating a response to a drug treatment in cancer patients, in general or for specific drugs (e.g. immunotherapies), specific technologies (e.g. PET and Imaging), and/or specific types of cancers (e.g. solid cancers), as recently reviewed (Rossi S et al., 2017; Subbiah V et al., 2017; Eleneen Y and Colen R R, 2017).

A first example of an effective therapeutic drug combination may involve the administration of Toll-like receptor agonists (TLR agonists) and other TLR ligands that are under clinical development, but whose activity depends from interferon-based mechanisms irrelevant for the therapeutic activity of a BO11X formulation, such as BO-112 formulation. Patients that are resistant or insensitive to any of such agents may be preferably treated by a BO-11X formulation, alone or in combination with another drug (e.g. a checkpoint inhibitor). Examples of such TLR ligands or agonists include small molecules, antibodies, or nucleic acid-based compounds that are TLR9 agonists (such as CpG-based nucleic acids), TLR4 agonists, TLR6 agonists, and or TLR8 agonists (see the review of TLR agonists under clinical development for cancer immunotherapy published in Smith S. et al. 2018).

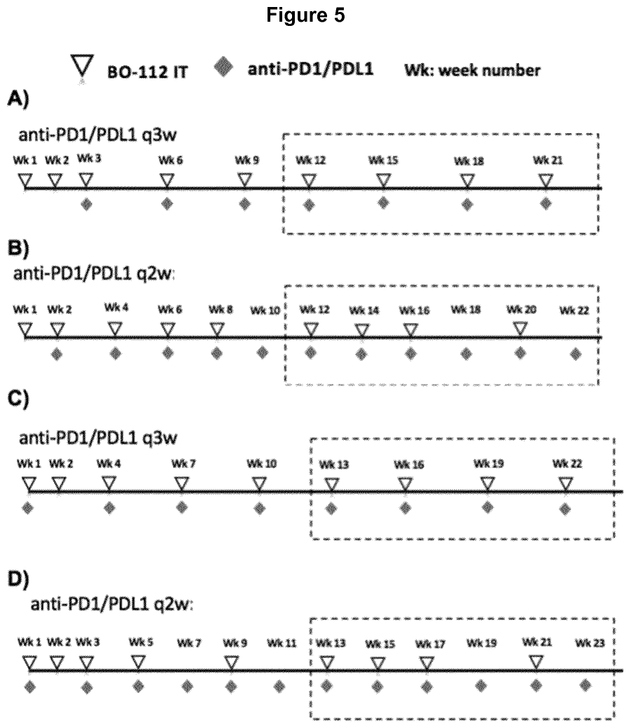

Another example of a therapeutic combination may involve the administration of anti-PD-1 and anti-PD-L1 antibodies (anti-PD1/PD-L1) whose efficacy as cancer immunotherapeutic agents may be improved (or simply made possible) by modifying an existing regimen for such drugs (involving regular intravenous injections of the antibody) with the inclusion of the administration of a BO-11X formulation, such as a BO-112 formulation, preferably injected intratumorally into one or more tumor lesions, one or multiple times). As also shown in FIG. 5, the disclosed BO-112 formulation may be initiated either before the beginning of a standard anti-PD1/PD-L1 clinical protocol or concomitantly with the anti-PD1/PD-L1 protocol (i.e. by administering anti-PD1/PD-L1 either every 3 weeks or every 2 weeks). The administration of the BO-112 formulation, in particular by intratumoral injection, may be performed every week (or daily) when the anti-PD1/PD-L1 antibody is also administered. Alternatively, the drug regimen may include specific weeks in which only one treatment (either BO-11X intratumoral injection or anti-PD1/PD-L1 intravenous injection) is performed. The drug regimen may also include a maintenance cycle in which the two drugs are alternatively or concomitantly administered over a number of weeks (e.g. 8, 10, 12, or more weeks) and such cycle is repeated 2, 3, 4 or more times.

Cancer patients that are identified as suitable candidates for BO-11X treatment (as first line or later line of treatment) may be selected (or excluded) for such treatment, or identified to be preferably treated with a BO-11X formulation since presenting the appropriate combination of clinical parameters and/or biomarkers and/or not responding to standard-of-care cancer therapies (such as radiotherapy or chemotherapy) or drugs that target to specific cancer antigens, immune checkpoints, cells, or biological pathways. These drugs may be of a different chemical nature, such as a small molecule (e.g. inhibitors of kinases or other enzymes), peptides (such as cancer vaccines) antibodies, (e.g. directed against PD-1, PD-L1, CTLA4, or CD20), cell-based products (e.g. adoptive cell transfer), or nucleic acids (e.g. targeting TLR9 or other receptor, DNA vectors). With respect to antibodies directed to cancer antigens, exemplary antigens are those approved (or under validation and review) by regulatory authorities including the EMA (in Europe) or the FDA (in USA), in addition to targeting cell surface proteins, in particular for treating solid tumors, and characterized by a therapeutic activity and/or a clinical use that may be improved by a combined administration with a pharmaceutical composition having adjuvant-like and immunostimulatory activities, including BO-11X formulations. Examples of such antigens are PDGFRalpha, Her2, EGFR, VEGFR2, RANKL, EpCAM, VEGFA, CEA, CD40, and CD25 (Corraliza-Gorjón I et al., 2017).

The present invention further relates to uses and methods applicable to the pharmaceutical compositions comprising nanoplex particles that are disclosed in PCT/EP2016/078078 and PCT/EP2017/079688 with respect to composition components, manufacturing of the composition, biophysical features and biological activities of the composition, and other criteria. In particular, the BO-11X formulation used in accordance with the present invention relates to a composition comprising particles wherein: (i) each particle comprises a complex of at least one double-stranded polyribonucleotide, or a salt or solvate thereof, and at least one polyalkyleneimine, or a salt and/or solvate thereof; (ii) at least 95%, or at least 90%, of said particles has a diameter of less than or equal to 600 nm, preferably, less than or equal to 300 nm (for example, between 140 and 250 nm); and (iii) said particles have a z-average diameter of less than or equal to 200 nm, preferably less than or equal to 150 nm, in particular, as measured according to ISO 22412:2017 (or using a subsequent update of this ISO standard).

In a preferred embodiment, the BO-11X formulation that is used according to present invention relates to an aqueous composition comprising particles wherein: (i) each of said particles comprises a complex of at least one double-stranded polyribonucleotide, or a salt or solvate thereof, and at least one linear polyalkyleneimine, or a salt and/or solvate thereof, wherein said double-stranded polyribonucleotide is polyinosinic-polycytidylic acid [poly(I:C)] and average molecular weight of linear polyalkyleneimine is between 17 and 23 kDa; (ii) at least 90% of said particles has a mono-modal diameter distribution below 300 nm; (iii) said particles have a z-average diameter of less than or equal to 200 nm, as measured according to ISO 22412:2017; and (iv) said composition has a zeta potential equal or superior to 30 mV, preferably between 35 and 50 mV, according to ISO 13099-2:2012.

In a further preferred embodiment, the BO-11X formulation that is used according to present invention relates to an aqueous composition comprising particles wherein: (a) each particle comprises a complex of at least one double-stranded polyribonucleotide, or a salt or solvate thereof, and at least one polyalkyleneimine, or a salt and/or solvate thereof, wherein the double-stranded polyribonucleotide is polyinosinic-polycytidylic acid [poly(I:C)], wherein at least 60% of the double-stranded polyribonucleotides have at least 850 base pairs, at least 70% of the double-stranded polyribonucleotides have between 400 and 5000 base pairs, and between 20% and 45% of the double-stranded polyribonucleotides have between 400 and 850 base pairs; and the polyalkyleneimine comprises at least 95% polyethyleneimines, wherein the average molecular weight of the polyalkyleneimine is between 17 and 23 kDa, with a polydispersity index that is equal or inferior to 1.5, and the ratio of the number of moles of nitrogen of the polyalkyleneimine to the number of moles of phosphorus of the double-stranded polyribonucleotide in the composition is between 2.5 and 5.5; and (b) the particles have a z-average diameter measured according to ISO 22412:2008 of between 30 nm and 150 nm; and (c) wherein at least 99% of the particles have a diameter distribution below 600 nm.

The composition in the latter BO-11X formulation embodiment contains at least 95% of the particles having a diameter of less than or equal to 400 nm. Moreover, this composition has a zeta potential of between 35 and 45 mV, measured according to ISO 13099-2:2012 (or per the later update of this ISO standard). The nanoplex particles in this composition contains polyethyleneimines that are preferably water-soluble, linear polyalkyleneimines, whereby the ratio of the number of moles of nitrogen of the linear polyalkyleneimine to the number of moles of phosphorus of polyinosinic-polycytidylic acid [poly(I:C)], in the composition is between 2.5 and 3.5.

The present invention also relates to uses and methods applicable to BO-11X formulations that comprise particles wherein: (i) each of said particles is formed by making a complex of at least one double-stranded polyribonucleotide, or a salt or solvate thereof, and at least one linear polyalkyleneimine, or a salt and/or solvate thereof, wherein said double-stranded polyribonucleotides are poly(I:C)] molecules and average molecular weight of said linear polyalkyleneimine is between 17 and 23 kDa; (ii) at least 90% of said particles has a mono-modal diameter below 300 nm; said particles have a z-average diameter of less than or equal to 200 nm, as measured according to ISO 22412:2017; and (iii) said composition has a zeta potential equal or superior to 30 mV, preferably between 35 and 45 mV, according to ISO 13099-2:2012; wherein said particles are formed at the ratio of the number of moles of nitrogen of said polyalkyleneimine to the number of moles of phosphorus of said double-stranded polyribonucleotide in said composition being equal to or greater than 2.5.

The present invention also relates to the use of BO-11X formulations that may be further defined on the basis of features measured in the aqueous composition, such as a pH of between 2 and 4, an osmolality of between 200 and 600 mOsm/kg, a zeta potential of between 35 mV and 40 mV or between 40 mV and 45 mV (according to ISO 13099-2:2012), and a concentration of one double-stranded polyribonucleotide equal to or greater than 0.5 mg per mL of the total volume of the composition. These BO-11X formulations may be used following lyophilisation and reconstitution of the aqueous composition in accordance to the criteria and features defined above.

Further preferred compositions of the provided BO-11X formulations to be used in methods and regimens of the present invention are described in PCT/EP2016/078078 and PCT/EP2017/079688 with respect to the ranges of sizes applicable to nanoplex particles disclosed herein. These compositions can be further defined on the basis of amount and size of the poly(I:C) molecules, in particular: (i) at least 40% of the double-stranded polyribonucleotides comprised in said particles have at least 850 base pairs, and at least 50% of the poly(I:C) molecules comprised in said particles have between 400 and 5000 base pairs; and/or (ii) between 5% and 60% of poly(I:C) molecules having less than 400 base pairs, between 15% and 30% of poly(I:C) molecules having between 400 and 850 base, between 10% and 70% of poly(I:C) molecules having between 850 and 5000 base pairs, and between 0% and 10% of poly(I:C) molecules having more than 5000 base pairs.

As described in PCT/EP2016/078078 and PCT/EP2017/079688, these compositions have been successfully optimized for pharmaceutical manufacturing and clinical use, in particular by identifying the optimal and more reproducible combinations of components, physical and/or chemical criteria, and related numerical ranges (applicable to either the particles or the compositions) for the desired uses and methods, such as: (a) said particles have a median diameter (D50%) of between 75 nm and 150 nm (and preferably at least have a median diameter of 85+/−20 nm); (b) the polydispersity index of said particle diameter is comprised between 0.2 and 0.3; and (c) the composition having a pH 3.0+/−0.2 and an osmolality of between 300 and 310 mOsm/kg.

In addition, the present invention further relates to a composition, for example, the BO-11X formulations or compositions disclosed herein, containing nanoplex particles in which the polyinosinic-polycytidylic acid [Poly(I:C)] and/or the polyethyleneimine (PEI) is fluorescently labeled. The fluorescent label (or fluorophore) can be any commercially available label used for modifying such particle components and, when both particle components are labeled, the fluorescent label may be different with respect to the wavelengths of excitation and/or emission between Poly(I:C) and PEI molecules. Examples of fluorescent groups that can be used as labeling agents are available from a variety of commercial providers (for example, Mirus Bio LLC, Chroma, IDT, LumiProbe, or Sigma), together with the relevant technical information and kits for generating such labeled preparations. A preferred fluorophore useful for labeling the polyethyleneimine component of the nanoplex particles is Cy5. A preferred fluorophore useful for labeling the poly(I:C) molecules comprised in the nanoplex particles is Rhodamine.

The presently disclosed Examples provide additional details about suitable combinations of fluorescently labeled and unlabeled components that are separately prepared and then used for preparing labeled BO-11X formulations (BO-11XL) in accordance with the protocols and methods disclosed in PCT/EP2016/078078 and PCT/EP2017/079688, having biophysical and biological features comparable to the BO-11X formulations described therein. BO-11XL formulations may comprise a variable percentage of labeled nanoplex particles (e.g. at least 1%, 5%, 10%, 20%, 50%, 70%, 90% or more of nanoplex particles with the formulation). BO-11X and BO-11XL formulations may be separately produced and compared using any appropriate model or, as applicable, mixed in various ratios in a combination (e.g., 1:1 or with either formulation present in a larger amount) before the actual use in a model. For instance, BO-11XL formulation can be used as a tracer within BO-11X formulation by adding a small percentage (20%, 10%, 5%, 1% or less) to a BO-11X formulation prior to use.

Similar to BO-11X formulations, BO-11XL formulations may further comprise at least one pharmaceutically acceptable carrier, organic solvent, excipient and/or adjuvant, either included in the particles themselves or added to the aqueous composition, for example, glucose added at a concentration of between 1 and 10% (weight/volume). Other types of BO-11X formulations are described in PCT/EP2016/078078, and PCT/EP2017/079688. The present compositions may further comprise at least one compound, and in particular a therapeutic compound, e.g. an organic compound, an inorganic compound, a nucleic acid (for example, non-coding RNA or RNA coding for proteins), an aptamer, a peptide or a protein, that may be included in particles (forming BO-11XLm formulations).

The presently disclosed Examples demonstrate how BO-11XL formulations can be used in cell-based models (using cell lines, primary cell preparations or co-culture systems such as organoids), animal models (e.g. relevant studying cancer pathology and cancer treatments), and/or ex vivo models (including tumor biopsies, PBMCs, or plasma samples) to define which cancer cells, immune cells, or other cells are capable of internalizing nanoplex particles such as those comprised in pharmaceutical compositions described in PCT/EP2016/078078 and PCT/EP2017/079688, and then presenting biological features relevant for a therapeutic use of the BO-11X formulations. Exemplary biological features that can be determined in these models by means of BO-11X and/or BO-11XL formulations include cytotoxicity, necrosis, differentiation, apoptosis, autophagy, (in)activation, migration outside or inside specific tissues or organs, proliferation, or other ones that such cells later exert in the human body (within the tumor, in lymph nodes, in blood, etc.) or by alternative mechanisms (such as changes in the combined secretion of chemokines, cytokines, interferons, etc., or in the expression of specific cell surface receptors, transcription factors, or enzyme involved in epigenetics or DNA repair). Exemplary models for validating such mechanisms in cell-based or animal models are described in PCT/EP2016/078078, PCT/EP2017/079688, and in the Examples herein. In particular, BO-11XL formulations allow demonstrating that a BO-11X formulation may be internalized not only by different cell types (such as cancer cells and immune/stromal cells) by, for instance, affecting their properties in a distinct manner, but BO-11XL formulations may also be identified and found to exert biological effects at specific locations distinct from the location where the composition was administered, for instance, within lymph nodes. These observations are useful for validating the therapeutic use of BO-11X formulations as a monotherapy or in a combination therapy where the effect of the BO-11X formulation in lymph nodes may be useful in a clinical setting.

The present invention further provides medical methods and uses that may combine different routes of administration of a BO-11X formulation (e.g. one or more intratumoral injections followed by one or more sub-cutaneous, intra-venous, intra-nodal, or intra-muscular injections over a period of one or more weeks) and/or the ex vivo exposure of human cells to the disclosed compositions, prior to re-administering the cells to the patient. In addition, in vitro and/or ex vivo studies designed to investigate an immune response to a BO-11X and/or BO-11XL formulation can show which drug regimens and methods of treatment involving the administration of a BO-11X (or BO-11Xm) formulation may trigger alternative biological mechanisms (interferon-independent and/or targeting immune cells, for example) that can be beneficially exploited for promoting therapeutically relevant events such as tumor cell death, enhanced local and/or systemic T cell immune response either directly (within injected tumors) or in distant tumors, and other mechanisms that may be useful for treating cancers that are recurrent, unresponsive or refractory to other therapies. The dose of the composition, in particular with respect to the content of double-stranded polyribonucleotides, can be adapted to each type of administration, drug regimen (e.g. highest for intratumoral or intrahepatic injection, lower for subcutaneous, intranodal or intramuscular injection, and even lower for treating cells ex vivo), and/or other drugs (when in combination therapies with another drug or in vaccination treatment regimens).

Other objects of the present invention relate to methods for evaluating the efficacy, the optimal drug regimen, the optimal therapeutic combination with another anti-cancer drug or standard-of-care protocol, and/or subjects presenting a strong response to treatment with BO-11X or BO-11Xm. These methods involve measuring the up- and down-regulation in the expression of panels of genes from selected cell types (such as immune cells and cancer cells) following exposure to a BO-11X composition and consequently applying appropriate means for improving therapeutic efficacy (e.g. for stratifying or selecting patients for further treatments, administering or not drugs targeting specific biological targets, and/or reducing or increasing the dosage of BO-11X and/or other therapeutic compositions).

Also provided herein are methods for making a pharmaceutical composition of BO-112 (and/or other BO-11X formulations, such as BO-11Xm formulations, BO-11XL formulations, BO-11XL formulations, or BO-112m formulations), including mixing a BO-11X formulation and one or more pharmaceutically acceptable adjuvant, diluent, carrier, or excipient thereof. Such components can be adapted for the specific medical indication (e.g. a solid cancer or a hematological cancer) and/or the administration means e.g. by injection (peritumoral, intratumoral, intraocular, intranodal, intrahepatic, intrapancreatic, intramuscular, or subcutaneous injection), by inhalation, topically, or orally. Additional embodiments relating to salts, pharmaceutical compositions and doses for BO-11X formulations that can be used according to the present invention are described in PCT/EP2016/078078, PCT/EP2017/079688, and reference literature such as in Remington's Pharmaceutical Sciences (edited by Allen, Loyd V., Jr; 22nd edition, 2012). BO-11X formulations may conveniently be presented in unit dosage forms and may be prepared by any of the methods known in the art of pharmacy. Such methods generally include bringing the therapeutic agents into association with a carrier, which constitutes one or more accessory ingredients, and may vary according to the particular method of treatment, the particular dosage form, and the mode of administration. Numerous factors that may modify the physiological action of the BO-11X and BO-11Xm formulations (e.g. body weight, gender, diet, time of administration, route of administration, rate of excretion, condition of the subject, drug combinations, genetic disposition and reaction sensitivities) can be taken into account by those skilled in the art. Administration of the formulations can be carried out continuously or in one or more discrete doses within the maximum tolerated dose, adapting, as needed, any other drug or standard-of-care treatment.

Optimal administration rates for a given set of conditions can be ascertained by those skilled in the art using conventional dosage administration tests. Individual doses of the agents described herein and/or pharmaceutical compositions of the present invention can be administered in unit dosage forms (e.g., tablets or capsules) containing, for example, from about 0.01 mg to about 1,000 mg of poly(I:C) molecules within the BO-11X formulation or BO-11Xm formulation, e.g. wherein said individual BO-11X formulation is formed by making a complex from about 0.01 mg to about 10 mg of poly(I:C)] preferably about 0.5 mg to about 2.5 mg of poly(I:C), more preferably about 1 mg to about 2 mg of poly(I:C), inclusive of all values and ranges there between. In some embodiments, the agents described herein and/or pharmaceutical compositions of the present invention are administered at an amount of from about 0.01 mg to about 1000 mg of poly(I:C) molecules within the BO-11X formulation daily, or from about 0.1 mg to about 10 mg daily, inclusive of all values and ranges there between. In accordance with certain embodiments of the invention, the agents and/or pharmaceutical compositions described herein may be administered, for example, more than once daily, about once per day, about every other day, about every third day, about once a week, about once every two weeks, or about once every three weeks. A suitable dosage of the agents and/or pharmaceutical compositions of the present invention is in a range of 0.1 to 10 mg of poly(I:C) molecules within the BO-11X formulation per subject (patient), preferably 0.5 to 2 mg of poly(I:C) molecules within the BO-11X formulation per subject, more preferably 0.6 to 1 mg per subject over an cycle of treatments. When a BO-11X formulation is administered by intranodal, intravenous, intrahepatic, intracutaneous or intratumoral injection, the total amount of composition can be adapted consequently.

The methods of treatment and uses of BO-11X formulations according to the present invention relate to treating or preventing a cell growth disorder characterized by abnormal growth of human or animal cells, preferably cancer, and most preferably solid cancers or lymphomas. In a further preferred embodiment, the methods of treatment and uses of BO-11X formulations as a pharmaceutical composition formulated (e.g. as an injectable, aqueous composition, optionally comprising a pharmaceutically acceptable carrier, excipient and/or adjuvant) and administered for the delivery of double-stranded polyribonucleotides to an organ or a tissue in a healthy state, presenting a disease related to a exogenous pathogenic agent (such a bacteria, a virus, and infections in general), or presenting an alteration due to a cell growth disorder characterized by abnormal growth of human or animal cells for instance, due to cancer (that is, involving tumorigenic transformation, metastasis, toxic compound), or a gynecological disorder characterized by abnormal growth of cells of the female mammal reproductive organs). Exemplary models for evaluating the methods or treatment and uses of the present invention are described in PCT/EP2016/078078, PCT/EP2017/079688, and in the Examples described below.

Preferably, the methods of treatment and uses of BO-11X or BO-11Xm formulations are intended for inducing (directly or indirectly) the death of one or more tumor cells or suppressing growth of the one or more tumor cells, and further including treating, reducing, ameliorating, or preventing cancer growth, survival, metastasis, epithelial-mesenchymal transition, immunologic escape or recurrence. In some embodiments, the BO-11X formulation (or a BO-11Xm formulation) and/or immune-modulating agent is used to treat cancers at various stages (e.g. Stage I, or Stage II, or Stage III, or Stage IV). By way of non-limiting example, using the overall stage grouping, Stage I cancers are localized to one part of the body; Stage II cancers are locally advanced, as are Stage III cancers. Whether a cancer is designated as Stage II or Stage III usually depends on the specific type of cancer. In one non-limiting example, Hodgkin's disease, Stage II indicates affected lymph nodes on only one side of the diaphragm, whereas Stage III indicates affected lymph nodes above and below the diaphragm. The specific criteria for Stages II and III therefore differ according to diagnosis. Stage IV cancers have often metastasized, or spread to other organs or throughout the body. Alternatively, the methods of treatment and drug regimens according to the present invention are used for adjuvant therapy, i.e. a treatment that is given in addition to the primary, main or initial treatment. By way of non-limiting example, adjuvant therapy may be an additional treatment usually given after surgery where all detectable disease has been removed, but where there remains a statistical risk of relapse due to occult disease. In some embodiments, the agents described herein are used as an adjuvant therapy in the treatment of a cancer.

In present invention, the cancer treated by the disclosed compositions, combinations, drug regimens, and related methods of administration is one or more of basal cell carcinoma, biliary tract cancer; bladder cancer; bone cancer; brain and central nervous system cancer; breast cancer; cancer of the peritoneum; choriocarcinoma; connective tissue cancer; cancer of the digestive system (including esophageal, stomach, colon, rectal or other gastrointestinal cancer); eye cancer; cancer of the head and neck; glioblastoma; hepatic carcinoma; hepatoma; intra-epithelial neoplasm; kidney, adrenal, or renal cancer; leukemia; liver cancer; lung cancer (e.g. small-cell lung cancer, non-small cell lung cancer, lung adenocarcinoma, and lung squamous carcinoma); melanoma; renal cell carcinoma; myeloma; neuroblastoma; oral cavity cancer (lip, larynx, tongue, mouth, and pharynx); pancreatic cancer; prostate cancer; retinoblastoma; rhabdomyosarcoma; cancer of the respiratory system; salivary gland carcinoma; skin cancer; squamous cell cancer; testicular cancer; thyroid cancer; uterine, endometrial, cervical, vulval, ovarian or other gynecological cancer; cancer of the urinary system; lymphoma including B-cell lymphoma, Hodgkin's and non-Hodgkin's lymphoma (NHL; including specific types such as low grade/follicular, small lymphocytic, intermediate grade/follicular, intermediate grade diffuse, high grade immunoblastic, high grade lymphoblastic, high grade small non-cleaved cell, or bulky disease NHL), mantle cell and AIDS-related lymphoma; chronic lymphocytic leukemia; acute lymphoblastic leukemia; Hairy cell leukemia; chronic myeloblastic leukemia; as well as other carcinomas and sarcomas; post-transplant lymphoproliferative disorder (PTLD), as well as abnormal vascular proliferation associated with phakomatoses or edema (such as those that associated with brain tumors). In some embodiments, the cancer is a biliary tract cancer. In some embodiments, the biliary tract cancer is selected from pancreatic cancer, gallbladder cancer, bile duct cancer, and cancer of the ampulla of Vater. In some embodiments, the cancer is liver cancer. In some embodiments, the cancer is colon cancer. In some embodiments, the biliary tract cancer is cholangiocarcinoma and/or an adenocarcinoma. Alternatively, the cancer may be listed among the list of rare diseases, being defined according to the criteria of incidence and/or prevalence as defined in Europe or USA, and indicated in the regularly updated lists made available in the website of organisations such as the International Rare Cancers Initiative (http://www.irci.info) or RareCare (http://www.rarecare.eu).

More preferably, the BO-11X formulations according to the invention are used in methods of treatment for treating solid tumors, such as carcinomas, gliomas, melanomas, or sarcomas. In particular, the BO-11X formulations are administered either systemically or more directly within or at a location near to the tumor such as at the margin of the tumor mass, in the surrounding epithelial cells, lymphatic or blood vessels (e.g. by intratumoral or peritumoral injection), or the abnormally growing cells of female mammal reproductive organs. The cancer may be a dormant tumor, which may result from the metastasis of a cancer. The dormant tumor may also be left over from surgical removal of a tumor. The cancer recurrence may, for example, be tumor regrowth, a lung metastasis, or a liver metastasis, wherein the methods of treatments and uses of the invention provides for reducing or blocking metastasis in distant sites that treated cancer (e.g. by intratumoral injection of BO-112 formulation) may otherwise have caused.

Additionally, macroscopic examination of organs and skin and microscopic, pathological analysis in either immune-deficient fully immune competent animal models may further indicate the efficacy of the methods and uses according to the present invention. The quantitative data that are generated in similar studies can be compared among the different experimental groups by using the appropriate statistical tests, with and without corrections for multiple testing, at the scope to evaluate which therapeutic (in particular anti-tumor) effects are provided by the administration of a BO-11X formulation, alone or in combination with another drug. Moreover, the methods of treatment and drug regimens according to the present invention may improve the effect of a BO-11X formulation in inducing local or systemic immunity by promoting immune cell memory allowing its use as single therapeutic agent, or in combination with immunomodulatory compounds, tumor-targeting agents, oncolytic viruses, adoptive cell transfer and other cell-based therapies, DNA- or peptide-based vaccines, or inhibitors of immunosuppressive metabolism, or other cancer-related metabolic pathway.

Furthermore, the disclosed compositions can be administered for supporting vaccines, cytokines, antigens, antibodies, chemical compounds, and other compounds having immunomodulatory activities for treating or preventing cancers (solid or not) or infection, for instance as adjuvant and/or for rescuing patients poorly responding or resistant to a drug, including agents for cancer immunotherapy, for altering cell metabolism and/or functions (preferably, in immune and/or cancer cells), for modulating DNA expression, replication and/or repair (including drugs that target epigenetic mechanisms), or for standard-of-care therapies (such as chemo- or radiotherapy, or vaccine-based therapies involving cancer or viral antigens). Such additional agent that is co-administered (subsequently, in any order) with a BO-11X (or BO-11Xm) formulation may improve a method of treatment with respect to the bioavailability, efficacy, pharmacokinetic/pharmacodynamic profiles, stability, metabolization, or other property of pharmaceutical interest not observed when treatment with each of an initial BO-11X formulation or another compound of pharmaceutical interest is administered alone.

The methods of treatment and regimens of the present invention can also involve the administration of an immune-modulating agent that is preferably an antibody including a monoclonal antibody and other antibody formats, or any other pharmaceutically available agent that binds a cell surface protein that control immune response, thus acting as a checkpoint inhibitor (CPI), which can block, reduce and/or inhibit PD-1 and PD-L1 or PD-L2 and/or the binding of PD-1 with PD-L1 or PD-L2.

Alternatively, the CPI can block, reduce and/or inhibit the activity of other immune checkpoint molecules such as LAG3, ICOS, CD137, CTLA-4, AP2M1, LAG3, OX-40, CD80, CD86, SHP-2, and/or PPP2R5A. As a further alternative, the CPI increases and/or stimulates CD137 (4-1BB) and/or the binding of CD137 (4-1BB) with one or more of a 4-1BB ligand and TRAF2. Other examples of a second therapeutic agent having immunomodulating properties include radiotherapy, chemotherapy CAR-T cells, cancer antigen vaccines, or agents that target regulatory T cells, metabolic enzymes, DNA repair and/or replication. The present invention also provides combining a BO-11X formulation with an immunomodulatory agent and/or with one or more common cancer treatment regimens (e.g. FOLFOX, FOLFIRI, radiation. photodynamic therapy or an antiproliferative agent such as doxorubicin, daunorubicin, mitomycin, actinomycin D, bleomycin, cisplatin, VP16, enedyine, taxol, vincristine, vinblastine, carmustine, and the like.

Exemplary cancer indications wherein the methods of treatment and drug regimens according to the present invention can be pursued by appropriately combining the administration of an agent inhibiting PD-1 (such an anti-PD-1 antibody) and a BO-11X formulation, with or without a standard-of care treatment (for instance, radiotherapy, as adjuvant), include, but are not limited to, melanoma, triple negative breast cancer, sarcoma, head-and-neck cancer, colorectal cancer, bladder cancer, renal cell carcinoma, liver metastasis, gastric cancer, prostate cancer, and hepatocellular carcinoma. Such indications may be also treated by administering a BO-11X formulation with an anti-PD-L1, an anti-CTLA4, or an anti-OX-40 antibody using an optimized drug regimen design.

In some embodiments, the immune-modulating agent targets one or more of PD-1, PD-L1, and PD-L2. Preferably, the immune-modulating agent is a PD-1 inhibitor. In some embodiments, the immune-modulating agent is an antibody specific for one or more of PD-1, PD-L1, and PD-L2. Such immune-modulating agent is an antibody, including the non-limiting examples of nivolumab, (ONO-4538/BMS-936558, MDX1106, OPDIVO), pembrolizumab (KEYTRUDA), pidilizumab (CT-011), MK-3475, BMS 936559, MPDL328OA. For example, the BO-11X formulation is combined with one or more of MPDL3280A (optionally with vemurafenib) and MEDI4736 (optionally with one or more of dabrafenib and trametinib) for the treatment of melanoma.

More particularly, the BO-11X (or BO-11Xm) formulations disclosed herein may be used for obtaining a synergistic therapeutic effect when administered with the second therapeutic agent, including reducing the regular dosage and/or frequency of administration of the second therapeutic agent (thus potentially reducing the need for additional medical intervention, an undesirable resistance to drugs, and/or a patient's overall discomfort). Moreover, the BO-11X (or BO-11Xm) formulations according to the invention, when administered with the second therapeutic agent, may allow treating patients that are resistant, insensitive, or presenting a poor clinical response to the second therapeutic agent, by overcoming any specific tumor resistance or escape mechanism (including mutations that alter specific genes, pathways, and/or response to drugs or endogenous compounds such as cytokines). Thus, the presently disclosed BO-11X (or BO-11Xm) formulations can be used in the form of a drug-rescuing or drug-sensitizing combination treatment, preferably for treating cancer. Such methods of treatment may involve administering the disclosed BO-11X formulation by intratumoral or peritumoral injection (within the tumor, at the margin of the tumor mass, in the surrounding epithelial cells, lymphatic or blood vessels) or other means that allow administering the BO-11X (or BO-11Xm) formulation directly within or in the proximity of cancer cells or organ comprising the cancer cells and the systemic administration of an immunostimulatory agent).

The BO-11X formulation (or a BO-11Xm formulation) according to the present invention can be also administered in methods of treatment and drug regimens involving a combination with cell-based therapies, wherein the BO-11X composition is either co-administered with the cell-based therapy directly to the patient, or wherein the BO-11X composition is used for treating cells obtained from a patient (from the blood or from a biopsy including cells within the tumor mass, at the margin of tumor mass, in surrounding epithelial cells, lymphatic or blood vessels). These cells, with or without a positive or negative selection for specific cell types, may be exposed to the BO-11X formulations in an appropriate laboratory setting for generating cells that, following this treatment, present markers, secrete proteins and/or expose antigens useful for further cancer treatment (e.g. cancer vaccines). Such autologous cells, again with or without further negative or positive selection, can be administered to the patient. During the later phases of treatment, the disclosed BO-11X formulations can be further administered to a patient by any appropriate means (for example, by intratumoral or, preferably, intra-muscular or sub-cutaneous injection). The ex vivo treatment of cells derived from a patient (e.g. dendritic cells) can be performed for a period of time of greater than one hour, preferably greater than three hours, more preferably greater than eight hours, even more preferably greater than twenty-four hours, or more, at a concentration lower than that required for intra-muscular or sub-cutaneous injection (preferably at 50%, more preferably at 25%, even more preferably at 10%, or much more preferably at 1% of such dose).

The present invention also provides methods of treatment and drug regimens involving the administration of a BO-11X formulation (or a BO-11Xm formulation) for increasing an immune response against a pathogen or other undesirable biological agent, and in particular for enhancing an anti-tumor immune response, potentially acting itself as an immune-modulating agent. Such an effect may be monitored by measuring a tumor-related immune response at the tumor site and tumor microenvironment (or in the bloodstream, other biological fluids, and tissues) at the level of relevant cell types or subpopulations (e.g. dendritic cells, T regulatory cells, T cells and/or NK cells) and/or of immunological biomarkers (e.g. chemokines, growth factors, cytokines, and their receptors). In particular, when the BO-11X formulations (or a BO-11Xm formulation) is clinically administered by intratumoral injection, apoptosis and/or necrosis is observed in the tumor, thus potentially promoting the presentation of tumor antigens to resident dendritic cells. A signaling cascade may lead also to the recruitment of immune cells, in particular CD4+ and CD8+ T cells into the tumor mass promoting an immune effect against the tumor, contributing to the cytotoxic effect of BO-11X. Otherwise, as described and shown in the Examples herein, the administration of the disclosed BO-11X formulations may induce changes in the absolute value and/or in ratio of specific immune cell populations, within tumors, lymph nodes, or both.

### EXAMPLES

### Example 1

### Preparation, Biophysical Characterization, and In Vitro Functional Validation of a Fluorescently Labeled BO-11X Formulation

### Materials & Methods

Main Reagents, Cell Lines, and Kits. BO-112 (GMP grade) was produced as described in PCT/EP2016/078078. PEI and Poly(I:C) preparations that were used for preparing the BO-11XL formulations herein, were purchased from Polyplus-Transfection® SA (France) and CARBOGEN AMCIS (Switzerland), respectively. Sulfo-Cy5 NHS ester was acquired from LumiProbe (Germany). LIVE/DEAD Red Dead Cell Stain kit were purchased from Thermo Fisher. The Label IT® Nucleic Acid Labeling Kit, TM-Rhodamine was acquired from Mirus Bio LLC (USA). The following cell lines were used and are available from ATCC or FROM Centra de Investigacion Medica Aplicada (Navarra, Spain): PANC02.03 (pancreatic cancer), B16-OVA (murine melanoma) and MC38 (murine colon carcinoma), NS47 (murine fibroblasts), RAW264.7 (murine macrophage), and DC2.4 (murine dendritic cells). Cell culture media and related materials were purchased from Gibco or Sigma-Aldrich. Fetal Bovine Serum (FBS) was bought from Capricorn Scientific (Germany). The 8-well and 15-well μ-Slides were acquired from iBidi (Germany). The assay was purchased from Promega. Precision Count Beads and ELISA MAX™ Mouse kits (TNF-alpha, IFNgamma) were purchased from BioLegend (USA). Calibration beads were purchased from Beckman Coulter.

PEI labeling. PEI was equilibrated to room temperature prior to any further handling. In a typical labeling experiment, a working solution of PEI was prepared by dissolving gently 25 mg of polymer (1.16×10-4 mmol/mL secondary amines, i.e. those present in the repeating monomeric unit) in 5 mL bicarbonate buffer 100 mM (pH=9.0, adjusted by adding adequate volumes of NaOH (aq.)) at room temperature. In parallel, 4.5 mg sulfo-Cy5 NHS ester was dissolved in 0.45 mL DMSO. Upon complete solubilization, 0.4 mL of the dye solution (containing 5-fold excess mol, targeting 0.2% mol secondary amines) was then added into the PEI solution and the mixture was allowed to stir (300 rpm) overnight at room temperature and protected from light. The labeled PEI product was purified via centrifugal ultrafiltration using Amicon filters (45 min at 4,500 g per wash) and washing against Milli-Q water (slightly acidic to ensure protonation of amines, hence polymer solubilization and recovery), until the fluorescence (measured via plate reader) and conductivity (measured via pH meter plugged to a conductivity probe) values of the wastewaters reached those of the Milli-Q water. The purified labeled PEI product was finally snap-frozen and freeze-dried (for at least 2 days to ensure complete removal of water). Polymer mass recovery was calculated as the amount of recovered polymer with respect to the initial mass and expressed as percentage. In order to prevent moisture, the freeze-dried polymer was stored in a tightly wrapped vial at −20° C. until use. The degree of functionalization was calculated by measuring the fluorescence intensity of a known concentration of fluorescently-labeled PEI product (Ex: 540/25, Em: 620/40 nm; Synergy HTX Biotek plate reader equipped with Gen5 software) using a calibration curve of Sulfo-Cy5 NHS ester dye to correlate emission to the molar concentrations of the fluorophore. Typical mass recovery value=70%. Typical degree of derivatization=0.1-0.15% mol.

Poly(I:C) labeling. The Label IT® kit was first warmed to room temperature and the ‘Labeling Reagent’ was briefly centrifuged to collect the lyophilized pellet. The pellet was reconstituted with 100 μL of ‘Reconstitution Solution’ and mixed thoroughly until suspended. In a typical labeling experiment, the following kit reagents were mixed with poly(I:C) (pIC) in the specified order: 700 μL RNAse-Free water, 100 μL ‘10X Labeling Buffer A’, 100 μL RNAse-Free water containing 5 mg/mL Poly(I:C), and 100 μL ‘Label IT® Reagent’, and incubated at 37° C. for 1.5 h protected from light. In order to minimize the effects of evaporation and maintain the right concentration of the reaction components, the vials were centrifuged half-way, i.e. after 45 min of incubation. The labeled poly(I:C) (pIC:Rho) was purified via centrifugal ultrafiltration using Amicon filters 3 (40 min at 5,000 g per wash, T=4° C.). The concentrated pIC:Rho was further washed with nuclease-free water until the fluorescence (measured via plate reader) and conductivity (measured via pH meter plugged to a conductivity probe) values of the wastewaters reached those of the RNAse-Free water. The pIC:Rho solution was then snap-frozen by dipping the vial in liquid nitrogen and freeze-dried (for at least 1-2 days to ensure complete removal of water). The recovered mass of pIC:Rho was recorded by weighting out the dry solid using a high precision balance and the mass recovery was calculated as the amount of recovered pIC:Rho (neglecting the contribution of the dye to the polymer mass) with respect to the initial pIC mass and expressed as percentage. In order to prevent moisture, the freeze-dried polymer was stored in a tightly wrapped vial at −20° C. until use.

Preparation of BO-11XL nanoplexes. Polymers were equilibrated to room temperature prior to any preparative operations and used to prepare either pIC:Rho-labeled or PEI-Cy5-labeled formulations using the protocol described in PCT/EP2016/078078, adjusting the pH where needed, and removing salts after centrifugal ultrafiltration similar to the protocols used for preparing the BO-112 formulation. All BO-11XL formulations were kept in sterile conditions at 4° C. until use.

Nanoplex characterization using Dynamic Light Scattering (DLS). The hydrodynamic diameter (Z-average size), surface charge (ζ-potential) and polydispersity index (PDI) of BO-112 and BO-11X nanoplexes were determined via DLS. Briefly, unlabeled and labeled nanoplexes were analyzed at 25° using a Zetasizer Nano ZS (Model ZEN3600, Malvern Instruments Ltd., UK) equipped with a solid state HeNe laser (λ=633 nm) at a scattering angle of 173° (pre-equilibration time=2 min; nanoplex concentration referred to pIC content=0.6 mg/mL; solvent=glucose 5% (w/v) (aq.)). The size and polydispersity data were calculated using the General-purpose algorithm. The electrophoretic mobility of the samples was converted into ζ-potential using the Smoluchowski equation.

General cell culture protocols. B16-OVA, DC2.4 and NS47 cell lines were grown in RPMI 1640, whereas MC38 and RAW264.7 cell lines were grown in DMEM. Media were supplemented with 10% (v/v) FBS, 2 mM Glu, 1% (v/v) sodium pyruvate, and 1% (v/v) P-S. In the case of NS47 and B16-OVA, media were supplemented with 0.25 μg/ml amphotericin B. Additionally, for NS47 and DC2.4 cell lines, media were supplemented with 1% (v/v) NEAA, 0.1% (v/v) 2-mercaptoethanol and 1% (v/v) HEPES. The FBS solution was heat-inactivated in house following standard procedures prior to culture media supplementation. All cells were cultured under standard conditions, i.e. 5% (v/v) CO₂ and 37° C., regularly tested for mycoplasma, and used at low passage numbers.

Uptake studies: Flow cytometry. Cells were seeded in 6-well plates (Corning), left to adhere and grow overnight, and finally incubated with 1 μg/mL BO-11XL(pIC:Rho) at 37° C. After specified incubation times, cells were washed with pre-warmed PBS and detached by incubation with pre-warmed Trypsin-EDTA for 5-10 min at room temperature. Uptake was determined on at least 5,000 live, individual cells with a CytoFLEX S (Beckman Coulter, Inc.) equipped with the CytExpert software (v2.0, Beckman Coulter, Inc.). Data were analyzed with Kaluza software (v2.1, Beckman Coulter, Inc.) after gating live and single events in the FSC-A/FSC-H (singlets) and FSC/SSC (live) windows, respectively. Untreated cells were used as an autofluorescence control in order to calculate the MFI n-fold for each time point, as well as the percentage of positive events (Laser: 561 nm, Fluorescent channel: 585/42 BP). DAPI was used to exclude dead cells (Laser: 405 nm, Fluorescent channel: 450/45 BP).

Uptake studies: Confocal microscopy. Cells were seeded at a low density in 15-well μ-Slides, left to adhere and grow overnight, and finally incubated with the treatment 1-10 μg/mL BO-11XL(pIC:Rho) at 37° C. After specified incubation times in glucose 5% (w/V) or media, cells were thoroughly washed with PBS, fixed with 4% paraformaldehyde (PFA) solution for 15 min at room temperature, washed again with PBS, and then permeabilized with 0.1% (v/v) TritonX-100 solution in PBS (10 min incubation at room temperature). Cell nuclei were counterstained with 1 μg/mL DAPI for 10 min at room temperature, then washed with PBS, and stored in the dark at 4° C. in a 1 mg/mL ascorbic acid/PBS solution.

MTS Assay. 5×10³ cells/well in 200 μL of growth medium were seeded in 96-well plates and left to adhere and grow overnight. Cell viability was measured via MTS assay after 24, 48, and 72 h incubation with 1 μg/mL BO-112 or BO-11XL preparations in complete medium. Untreated cells were used as negative control. At each time point, 20 μL of MTS solution were added to each well (following manufacturer's instructions) and the absorbance at 490 nm was recorded after a 1-4 hour incubation at 37° C. (Synergy HTX Biotek plate reader equipped with Gen5 software).

Results

The present invention provides a newly designed strategy for labeling the two components of the BO-112/BO-11X formulations, namely PEI and Poly(I:C), with two photostable and pH-insensitive fluorophores (over typical pH values commonly used for cell experiments), namely TM-Rhodamine and Cy5 (each having distinct fluorophore excitation and emission wavelengths that can be exploited in co-localization, multi-fluorescent studies for evaluating biodistribution and biological interactions).

The general procedures for preparing the Cy5-labeled PEI polymer is based on the reaction of sulfo-Cy5 NHS ester with the amine groups of the PEI, i.e. NHS ester reaction chemistry, for example using Sulfo-Cyanine 5 (Cy5) NHS ester. NHS esters are chemical groups that react with amine groups (reactivity increases in the order primary>secondary>tertiary amines) in slightly alkaline conditions (pH range 7.2-9.0) to yield stable conjugates via the formation of amide bonds. NHS esters are hydrolysed in a pH-dependent manner (the higher the pH, the quicker the hydrolysis), which in turn unfavourably contributes to less-efficient reactions and lower conjugation yields (due to competition of the hydrolysis product with the amine reaction). NHS ester reactions are commonly performed in buffers (e.g. phosphate or carbonate-based, HEPES, etc.) at room temperature or at 4° C. These procedures make use of a linear PEI polymer having a low molecular weight (MW range 17-23 kDa; average MW=20.5 kDa). For the conjugation of NHS fluorescent dyes, the reaction yield is determined via fluorimetry using a calibration curve of sulfo-Cy5 NHS ester to correlate the emission of the labeled polymer to the molar concentrations of the fluorophore, using a PEI sample of known concentration. This conjugation procedure can be readily applied to other commercially available fluorescent NHS esters fluorescent dyes.

The methods relating to the chemical (i.e. non-enzymatic) functionalization of poly(I:C) molecules (such the ones used for preparing BO-112 formulations), with TM-Rhodamine (Rho) according to the present invention, are based on a reactive alkylating agent featuring a strong nucleic acid binding capability (facilitated via electrostatic interactions) provided commercially by Mirus Bio LLC. The alkylation reaction can take place on reactive heteroatoms on any nucleotide of a DNA or RNA sample, without dramatically altering the starting nucleic acid or hindering downstream hybridization performance. These reactions are non-destructive to the nucleic acid starting material, and have been designed to target a lower degree of functionalization than that recommended by the manufacturer.

The resulting labeled polymer solutions were purified via centrifugal ultrafiltration against Milli-Q or RNAse-Free water, in the case of PEI or Poly(I:C) molecules, respectively, until the fluorescence and conductivity values of the wastewaters reached those of pure water. Both polymer products were purified and the majority of the initial mass was recovered (mass recovery for PEI:Cy5 and Poly(I:C):Rho was of 70% and 90-100%, respectively). In the case of PEI:Cy5, calculated degree of derivatization was 0.1% mol NH units. For poly(I:C) molecules, according to manufacturer's kit description, expected degree of derivatization was 1 Rhodamine label for every 4-12 bases.

Unlabeled BO-11X and BO-112 formulations were prepared as described in PCT/EP2016/078078 and PCT/EP2017/079688. Labeled BO-11X nanoplexes were prepared by following the same procedure, but using recovered fluorescently labeled polymers. All BO-11X formulations were kept in sterile conditions at 4° C. until use.

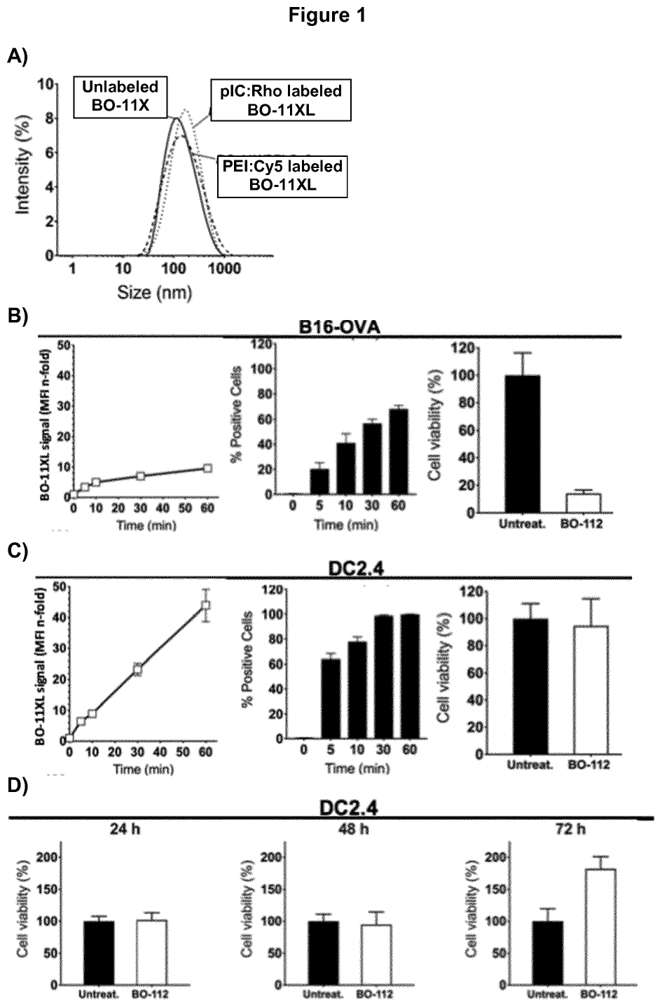

The physio-chemical characteristics of the resulting nanoplex particles in the BO-11XL formulations were then compared with those of BO-112 formulation using Dynamic light Scattering (DLS) and Atomic Force Microscopy (AFM), which confirmed highly similar morphological and biophysical characteristics for both BO-11XL and BO-112 formulations, (see FIG. 1A). For instance, AFM analysis revealed a similar spherical-like morphology and DLS comparable particle diameter, polydispersity index (around 0.3), and a Zeta potential (around 40 mv) between BO-112 and BO-11XL formulations.

In order to compare the biological properties of BO-11XL formulations against the BO-112 formulations, we first validated a straightforward in vitro cancer cell model based on Panc02.03, a human pancreatic cell line. The antitumoral effects of unlabeled and labeled BO-112/BO-11X nanoplexes were then evaluated using a MTS assay. MTS results revealed identical antitumoral effect for all formulations at 24, 48 and 72 hours.

The cell interactions of such validated, labeled BO-11XL formulations were then assessed with Panc02.03 cells. We first tracked the cellular uptake by following the signal corresponding to the carrier (i.e. PEI) or to the cargo (i.e. Poly(I:C) using flow cytometry (on live cells). The results returned similarly quick kinetics of uptake by monitoring either component. We also explored the entry mechanism for BO-11XL formulations by first blocking specific uptake pathways with pharmacological agents, then following the BO-11LX/PEI:Cy5 signal after a 10 min exposure. BO-11X appears taken up via chlatrin-dependent endocytosis (60% reduction for nocodazole-treated) in a process affected by an inhibition of endosome maturation (80% reduction for bafilomycin-treated) and macropinocytosis (30% reduction for EIPA-treated). This approach may be further developed to identify interactions, co-localization, and or mechanism of action of BO-11X formulation when administered alone or in combination with other therapeutic agents.

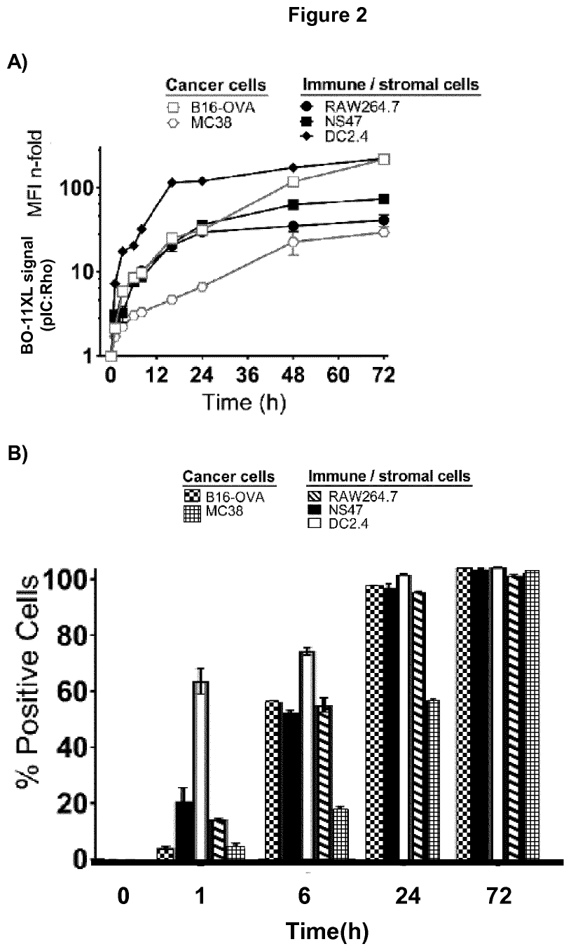

The characterization of BO-11XL formulations has been pursued in several reference murine cell lines such as B16-OVA (murine melanoma cell line), DC2.4 (murine dendritic cell line) and RAW264.7 (murine macrophage cell line), which were used as murine models of melanoma, dendritic cells and macrophages, respectively, that were cultured in standard conditions and tested at low passage numbers. As shown in FIG. 1 (at B-D), the uptake kinetics of the BO-11XL formulations was rapid and comparable across the three murine cell models, but only B16-OVA cells were notably killed by the BO-11X formulation, but not DC2.4 cells (RAW264.7 having similar properties). Indeed, the uptake of BO-11X by dendritic cells results in increased proliferative activities, suggesting that the BO-11X formulations according to the present invention may provide specific therapeutic benefit by targeting specific immune cells, such as subpopulations of dendritic cells within the tumor mass, in lymph nodes, and/or in the bloodstream. These findings can be further explored in human or murine cell lines, either from immune cells or cancer cells (eventually, in co-culture systems) in order to screen such cell lines with the BO-112 and/or BO-11XL formulations described herein, and further to define which cell types, using which mechanism, internalize the nanoplex particles within such formulations. Additionally, biological parameters such as cell viability, proliferation, differentiation, or activation may also be determined. Confirmatory findings were generated using a panel of cancer cells and immune/stromal cells (FIG. 2) that also did not show the specific effect of the BO-11X formulations on the metabolic activity (proportional to the cell viability and proliferation as measured through the colorimetric MTS assay at 72 hours post-treatment), with the cytotoxic effect of BO-11X formulations on cancer cells, or a lack of toxicity on fibroblasts and macrophages, or a proliferative effect on dendritic cells; this combined with an increased effect of BO-11X on the production of cytokines such as TNFalpha and IFNgamma, as measured via ELISA.

### Example 2

### In Vivo Functional Validation of a Fluorescently Labeled BO-11X Formulation

Materials & Methods

MC38-based cancer model. MC38 colon carcinoma cells were injected subcutaneously (5×105) into the upper right flank of 8- to 10-week-old female C57BL/6J mice (6-11 mice/group) on day 0. Tumors were measured twice per week using calipers, and the tumor volume was calculated (length×width2/2). When tumors reached a volume of 80-100 mm³ (day 0), the mice were then randomized into different groups of treatment according to the experimental protocol. Labeled BO-11XL(pIC:Rho) formulations (2.5 mg/kg, 100 μ1) were administered by intratumoral injection. The control group received intratumoral injections of 5% (v/v) glucose (BO-11X vehicle, identical volume) or unlabeled BO-112 (identical amount per dose). The mice were euthanized by CO₂ asphyxiation at different time points following intratumoral injection.

In vivo analysis. Biodistribution of BO-11XL(pIC:Rho) was determined using an IVIS SpectrumCT In Vivo Imaging System (PerkinElmer) with identical instrument settings (Filter: 535/580 nm). After acquiring whole animal images (side and supine (open) positions), tumors and lymph nodes were excised, and their fluorescence recorded. The Living Image Software (v4.2) was used for quantitative analysis. Fluorescent background subtraction was performed in all cases. Two independent experiments were performed.

In vitro lymph node cell analysis. Single cell suspensions were obtained by mechanical disaggregation of lymph nodes using a syringe plunger and a cell strainer. Before flow cytometry, harvested cells were treated first with pre-heated erythrocyte lysis buffer (NH4Cl) for 2 min at 37° C., then stained with an antibody cocktail (CD45:APC, CD19:FITC, CD3:APC-Cy7, CD4:BV510, CD8:PECy7, NK1.1:PECy5, diluted as per manufacturer's instructions) for 30 min at 4μ C. in dark conditions. Cells were then thoroughly washed with PBS and analyzed with a CytoFLEX S (Beckman Coulter, Inc.) equipped with the CytExpert software (v2.0, Beckman Coulter, Inc.). Data were analyzed with Kaluza software (v2.1, Beckman Coulter, Inc.). Calibration Beads and Precision Count Beads were used for compensation and to obtain absolute cell counts, respectively.

Results

The data generated using the BO-11XL formulations according to the present invention in described cell-based models can be exploited to establish animal models, wherein both the biological effects on specific cell types (in particular immune cells and cancer cells) and the anatomical distribution of BO-11X formulation can be characterized upon administration, in particular by intratumoral injection. For instance, the components of the BO-11XL formulations can be investigated to determine how the poly(I:C) molecules are associated (e.g. by cell internalization) to specific cells within the tumor mass, and/or how the poly(I:C) molecules diffuse to proximal or distal lymphatic networks or to other alternative locations relevant to the orchestration of immune responses. In particular, the fluorescently labeled poly(I:C) molecules can be detected after intratumoral injection of BO-11XL formulation in a syngeneic mouse cancer model based on murine colon adenocarcinoma MC38 cells that are subcutaneously implanted in C57BL/6 mice, or another relevant cancer mouse model, together with or separately from other drugs, such as an anti-PD-1 antibody having comparable qualities to those used in clinical activities.

The intratumoral injection of the BO-11XL formulations according to the present invention within these induced tumor lesions can be followed using the ex vivo detection of fluorescence intensity within the murine tissues in a time-dependent manner up to 48 hours post-treatment. The tumor mass and lymph nodes are excised and scanned separately in groups of mice sacrificed at different time points. The former type of samples can be frozen and sectioned, and immunofluorescence will be used to determine the cellular localization of labeled poly(I:C) molecules among the heterogeneous cell populations within the tumor mass. The latter sample types can be disaggregated to create single-cell suspensions that can be subjected to flow cytometry analysis in order to identify various biological features of the cell types and/or variations in cell sub-populations that can be defined as a consequence of BO-11XL treatment compared to a relevant control (e.g. tumors that are injected to vehicle only, free PEI, or free labeled poly(I:C) molecules, or not injected at all). This analysis may be also extended to DNA, RNA, and/or proteins that are extracted from the tumors and the lymph nodes to determine any positive (or negative) correlation across the localization of fluorescently labeled poly(I:C) molecules, gene expression data (in particular by focusing the analysis to specific expression profiles and any “signatures” associated with therapeutically relevant response, such as an immune response targeting cancer cells or pathogens), and any other relevant biological read-outs.

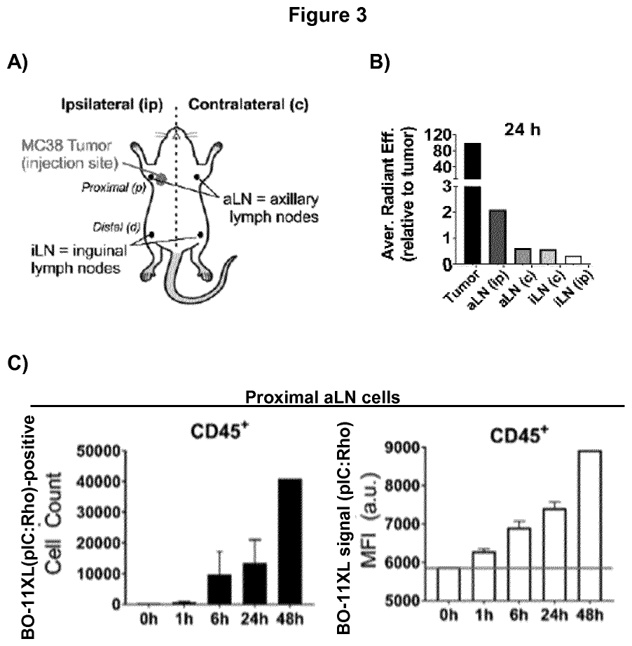

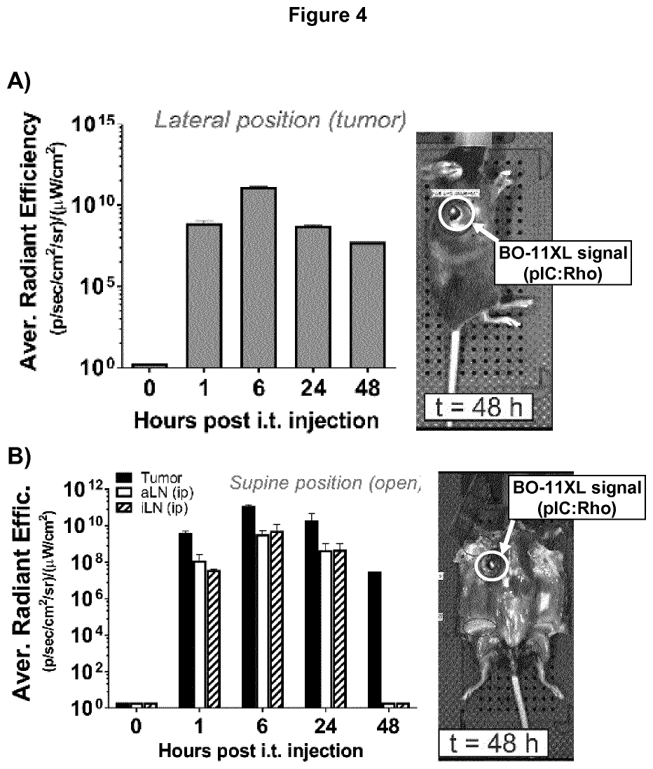

Additional confirmatory data was generated by injecting BO-111XL(pIC:Rho) into MC38-induced tumor and then analysing the distribution of the labeled Poly(I:C) molecules (being either still comprised in complexes with PEI or detached from PEI) within the injected tumor and main lymph nodes, either ex vivo by extracting the relevant tissues, isolating the cells, and pursuing flow cytometry analysis for identifying fluorescently labeled cells or increase of immune cells present in lymph nodes (FIG. 3), or alternatively, by performing an in vivo analysis of fluorescence at specific locations and positioning (FIG. 4).

In addition, the site(s) of the tumor lesion(s) used for intratumoral injection may also compared to identify which cells internalizing the disclosed BO-11X and BO-11XL formulations (or otherwise sensitive to the exposure to such formulations in vivo) confer a specific benefit by decreasing the injected tumor lesion (such a skin lesion, a subcutaneous nodule, a lymph node) as well as any distant, non-injected lesion (e.g. in a visceral organ such as liver or lung). This potentially differential effect may require further evaluation not only by analysing the relevant tissues, but also through an analysis of circulating cells (in particular dendritic cells, macrophages and other cells involved in immune responses) and proteins (chemokines, interferons and cytokine in general) within blood samples.

### Example 3

### Characterizing Regimens and Methods of Treatment Involving the Administration of a BO-11X Formulation in Clinical Studies, in Particular for Combined Drug Treatments

Materials & Methods

Nanostring® analysis. The analysis of the expression of specific gene signatures was performed after an extra informed consent form that is required for genetic testing was signed by enrolled patients in the clinical study “Exploratory Study of BO-112 in Adult Patients With Aggressive Solid Tumors” (NCT02828098). NanoString nCounter Technology was used for the expression assessment with the nCounter PanCancer Immune Profiling Panel, a 770-plex gene expression panel designed to measure both the adaptive and innate immune in solid and liquid cancer types in humans (Cesano J, 2015). Total RNA from tumor biopsies was isolated from the same patient before and after the injection of the disclosed BO-112 formulation. The tumor biopsies were rinsed in RNAlater (Thermo Fisher Scientific, Waltham, USA) immediately following their collection to preserve the integrity of the nucleic acids. The total RNA was isolated by Trizol (Thermo Fisher Scientific, Waltham, Mass. USA), following the manufacturer instructions, and the integrity/quality assessed by the Bioanalyzer technology (Agilent, Santa Clara, USA). The tests were performed at the Laboratory of Translational Oncology (Hospital General Universitario Gregorio Marañón, Madrid, Spain), and the bioinformatic analyses at Dreamgenics (Oviedo, Spain) following manufacturer instructions (Nanostring, Seattle, USA). NanoString counts were extracted using NanoString nCounter Software. Counts normalization was performed using RUVSeq R package, which detects unwanted experimental variation, such as hidden batch effects, and applies a normalization based on housekeeping genes (Risso D et al., 2014). Differential expression analysis of genes included in the nCounter PanCancer Immune Profiling Panel was performed with DESeq2 algorithm (Love M et al., 2014). P-values (-log10) and fold-change (log2) from the differential expression analysis between conditions were represented using volcano plots.

Results

PCT/EP2016/078078 and PCT/EP2017/079688 each describe a number of approaches for determining the clinical utility of a BO-11X formulation, such as BO-112 biological and therapeutic activities that can be observed in clinical studies, in particular following intratumoral administration of a BO-112 formulation. The combination of a BO-11X formulation with an anti-PD-/PD-L1 therapy can be adapted to different drug regimens (summarized in FIG. 5), and associated to the analysis of biological features that are measured prior, during, and after performing the clinical protocol. Such criteria can be measured in the bloodstream, in biopsies, and other biological samples obtained from the patients at distinct time points, in parallel to the evaluation of clinical parameters such as the size of treated and untreated tumor lesions, metastasis in other locations, and other clinical criteria that are defined under standardized systems including RECIST or irRC, in general or with reference to other treatments.

In particular, BO-112 has been administered combined with anti PD-1 in patients with solid tumors primarily resistant to anti-PD-1 (Nivolumab or Pembrolizumab). A lesion >1 cm amenable to Intratumoral injection was required to inject BO-112 (1 mg)×2 or 3 doses (qw) before continuing the previous anti PD-1 combined with BO-112, until progression, limiting toxicity or up to 1 year. Pre- and post-BO-112 biopsies from the injected lesion was analysed for necrosis, apoptosis, and immune infiltrate. Among the treated patients, partial response (PR) was observed in specific patients affected by melanoma and renal carcinoma, while other patients presented stable disease (SD). In this clinical study, the disclosed BO-112 combined with anti-PD-1 showed a manageable safety profile, direct antitumor effects and innate and adaptive immune system activation, suggesting a significant potential to reverse resistance to anti-PD1 treatment, with or without concurrent treatment by radiotherapy.

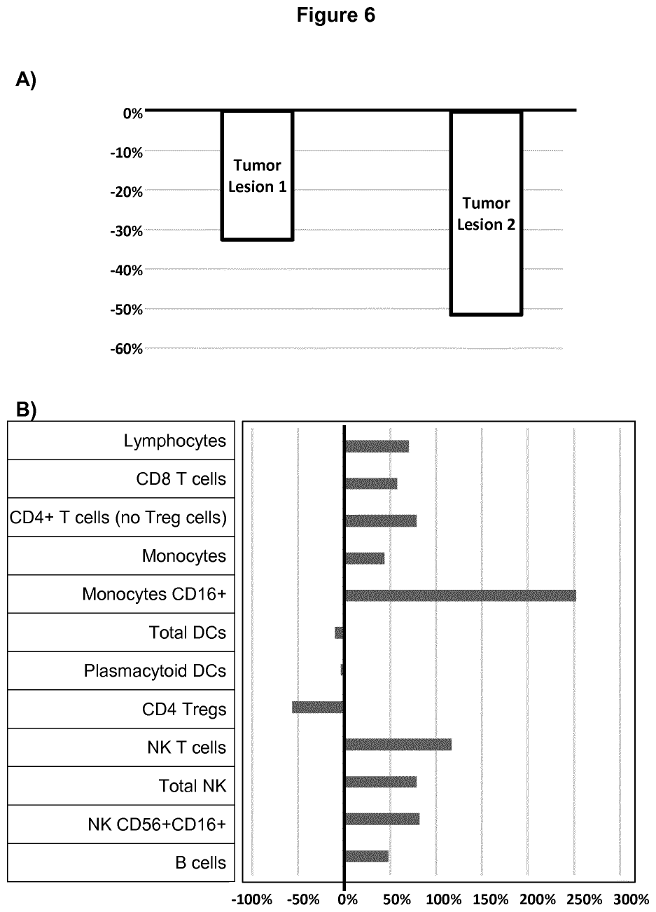

An exemplary analysis of BO-112 treatment was performed in a patient affected by renal cell carcinoma (FIG. 6). The patient was first treated with sunitinib, cabozantinib, everolimus, and then treated with nivolumab, and subsequently with a BO-112 formulation (administration by injecting supraclavicular lymph node). This treatment regimen led to significant necrotic effects in the injected lesions (with an increased presence of CD8-positive cells and specific macrophage sub-populations) and a decrease in the size of also non-injected, left & right para-aortic lymph nodes. This treatment paradigm has also been studied in tumor biopsies using immunohistochemistry and in circulating cells using flow cytometry, allowing the detecting of major changes in specific immune cell populations. This approach can be extended to clinical studies in which the patients are treated by applying one of the drug regimens depicted in FIG. 5 for an anti-PD-1/PD-L1 drug combination (or in combinations of BO-11X formulations with another immunomodulatory drug) to determine which clinical parameters are more regularly affected in a negative or positive manner, and potential biological features also consequently affected by the drug regimen, in particular with respect to how a direct effect on cancer cells and indirect, immune effect on tumor can be identified and possibly improved by selecting drug compositions, drug regimens, and drug combinations.

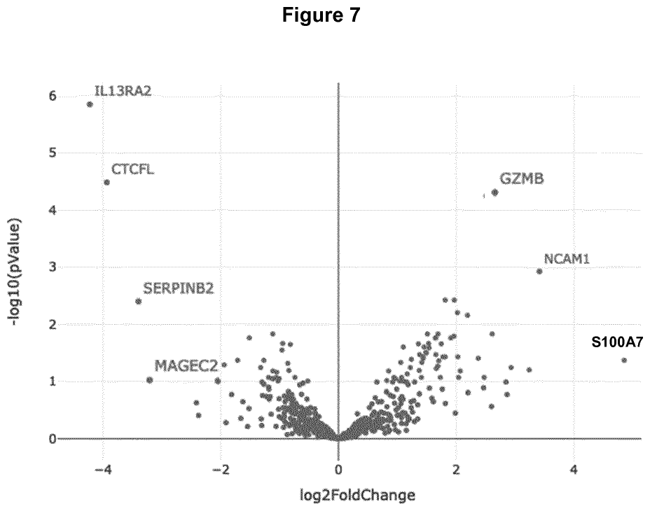

A further analysis in this clinical population can be pursued using Nanostring technology, a highly sensitive technology permitting the rapid parallel detection and quantification of mRNA found in cell samples for a panel of genes having been characterized as indicating the activation of specific cells, in particular with respect to an immune response, and enabling multiplexed gene expression analysis of clinical samples from patients (Veldman-Jones M et al., 2015). When tumor biopsies from a cohort of patients are analysed following treatment with an intratumoral injection of BO-112, and comparing patients that have shown, as best responses, PR+SD versus PD, the Nanostring analysis can be presented as a Volcano plot where those genes that are specifically up-regulated or down-regulated by BO-112 with higher statistical significance are separated from others in the top right and left areas of the graph (FIG. 7). Using this approach, the expression of human genes such as NCAM1 (Neural cell adhesion molecule 1), GZMB (granzyme B), and S100A7 (protein S100 calcium-binding protein A7) are specifically and significantly up-regulated in clinical samples derived from patients with PR and SD, as best responses, following BO-112 administration. In parallel, the opposite effect has been detected for genes such as IL13RA (IL-13 receptor subunit alpha-12, or CD213a2), CTCFL (Cancer/testis antigen 27, CT27), SerpinB2 (Plasminogen activator inhibitor 2, PAI-2), and MAGEC2 (Melanoma-associated antigen C2, Cancer/testis antigen 10, CT10). In this manner, a subset of genes can be used as a guide for defining the BO-112 administration effects and the biological readout for comparison with the clinical effects within tumors, lymph nodes, or other locations.

More generally, as exemplified in FIG. 8, the therapeutic regimens involving the administration of a BO-11X formulation according to the present invention can be adapted to the response observed in specific cancers or populations of patients. Moreover, the biological attributes measured prior to the actual treatment with the disclosed compositions that appear positively correlated with therapeutic effects may be then used for establishing assays and protocols for stratifying populations of patients and determining those would receive most therapeutic benefit by the BO-11X administration, alone or in combination with another drug (e.g. an anti-PD-1 antibody). The disclosed BO-11XL formulations can be also used in this manner by testing cells from the patients ex vivo and determining parallel effects of these formulations.

### REFERENCES

Cesano J, J Immunother Cancer. 2015; 3: 42.
Corraliza-Gorjon I et al.,. Front Immunol. 2017;8:1804.
Eleneen Y and Colen R R, Adv Exp Med Biol. 2017; 995:141-153.
Kadam, R N et al., Braz. J. Pharm. Sci. 2015; 51: 255-263.
Love M et al., Genome Biol. 2014; 15: 550.
Marquez Rodas I et al., Annals of Oncology 2018; 29 (suppl_8), mdy424.049,
Patel M C et al., Future Virol. 2014; 9:811-829.
Risso D et al., Nat Biotechnol. 2014; 32: 896-902.
Rossi Set al., EurJ Nucl Med Mol Imaging 2017; 44: 2310-2325.
Smith S. et al., Oncolmmunol. 2018; 7: e1526250.
Subbiah V et al., Diagnostics 2017; 7: E10.
Tormo D et al., Cancer Cell. 2009; 16: 103-14.
Veldman-Jones M et al., Cancer Res. 2015; 75: 2587-93.

1-31. (canceled)

32. A method for determining one or more drug regimens for the treatment of cancer, comprising:
(a) obtaining a biological sample from a subject presenting cancer;
(b) identifying a drug effect in the biological sample by detecting a clinical response in the subject following administration of a first therapeutic composition, alone or in combination with a second therapeutic agent; and
(c) determining the one or more drug regimens for treating the cancer in the sample based on the drug effect;
wherein the first therapeutic composition is an aqueous composition comprising a formulation of one or more nanoplexed particles, each respective nanoplexed particle optionally comprising a fluorophore label, wherein:
(i) each respective particle in the one or more nanoplexed particles comprises a complex of at least one double-stranded polyribonucleotide, or a salt or solvate thereof, and at least one polyalkyleneimine, or a salt and/or solvate thereof,
wherein the double-stranded polyribonucleotide is polyinosinic-polycytidylic acid [poly(I:C)],
wherein at least 60% of poly(I:C) molecules have at least 850 base pairs, at least 70% of poly(I:C) molecules have between 400 and 5000 base pairs, and between 20% and 45% of poly(I:C) molecules have between 400 and 850 base pairs; and the polyalkyleneimine comprises at least 95% polyethyleneimines, and
wherein the average molecular weight of the polyalkyleneimine is between 17 and 23 kDa, with a polydispersity index that is equal to or below 1.5, and the ratio of the number of moles of nitrogen of the polyalkyleneimine to the number of moles of phosphorus of the double-stranded polyribonucleotide in each respective nanoplexed particle in the aqueous composition is between 2.5 and 5.5;
(ii) each respective particle in the one or more nanoplexed particles has a z-average diameter measured according to ISO 22412:2008 of between 30 nm and 150 nm; and
(iii) at least 99% of the one or more nanoplexed particles have a diameter distribution below 600 nm.

33. The method according to claim 32, wherein the first therapeutic composition is an aqueous composition comprising particles wherein:
(i) each respective particle in the one or more nanoplexed particles is formed by a complex of at least one poly(I:C) molecule, or a salt or solvate thereof, and at least one linear polyalkyleneimine, or a salt and/or solvate thereof, wherein the average molecular weight of the linear polyalkyleneimine is between 17 and 23 kDa;
(ii) at least 90% of the one or more nanoplexed particles has a diameter below 300 nm and each respective particle has a z-average diameter of less than or equal to 200 nm measured according to ISO 22412:2017; and
(iii) the aqueous composition has a zeta potential equal to or above 30 mV, measured according to ISO 13099-2:2012; wherein the ratio of the number of moles of nitrogen of the polyalkyleneimine to the number of moles of phosphorus of the double-stranded polyribonucleotide of each respective nanoplexed particle in the aqueous composition is equal to or greater than 2.5.

34. The method of claim 32, wherein the second therapeutic agent is an anti-PD-1 or an anti-PD-L1 antibody.

35. The method of claim 32, wherein the one or more drug regimens for the treatment of cancer comprises radiotherapy.

36. The method of claim 32, wherein the cancer is selected from melanoma, triple negative breast cancer, sarcoma, head-and-neck cancer, colorectal cancer, bladder cancer, renal cell carcinoma, liver metastasis, gastric cancer, prostate cancer, and hepatocellular carcinoma.

37. The method of claim 32, wherein each respective nanoplexed particle is labeled with a fluorophore (BO-11XL).

38. The method of claim 37, further comprising in step (b) measuring the uptake of the one or more labeled nanoplexed particles into one or more cells present in the biological sample to identify a drug effect.

39. The method of claim 32, wherein the clinical response in step (b) is defined according to an analysis of the biological sample of step (a) for the presence of biomarkers, cancer antigens, immune cells, or clinical criteria selected from tumor burden, stage of the tumor, amount of metastasis, and tumor recurrence.

40. The method of claim 32, wherein the clinical response in step (b) is defined according to an analysis of the biological sample of step (a) for the presence of immune cells and cancer cells.

41. The method of claim 32, wherein the drug effect detected in step (b) is determined based on a corresponding assessment criteria of the clinical response for the subject according to RECIST, irRC, or PERCIST standardized criteria.

42. The method of claim 32, wherein the drug effect detected in step (b) corresponds to a decrease in size of injected and non-injected tumor lesions.

43. The method according to claim 42, wherein the drug effect detected in step (b) corresponds to any of the following, or a combination thereof:
the presence or absence of specific immune cells or populations of cancer cells or subpopulations thereof,
changes in the expression or activity level of genes and pathways for antigen presentation, or
the presence or activity of cross-presenting dendritic cells.

44. The method of any claim 32, wherein the drug regimen comprises:
administration of the first therapeutic composition alone,
administration of the first therapeutic composition in a combination with the second therapeutic agent performed on a daily or weekly basis,
administration of the first therapeutic composition is performed during a week without the second therapeutic agent and vice versa, or
administration of the first therapeutic composition alternatively or concomitantly on a weekly basis as a maintenance cycle.

45. The method according to claim 32, wherein the biological sample is a blood sample or a tumor biopsy derived from the subject.

46. A method for treating cancer comprising administering to a subject in need thereof a drug regimen defined according to claim 32.

47. A method for treating cancer comprising administering to a subject in need thereof a drug regimen defined according to claim 33.

48. A method for identifying a subject to be treated with the first therapeutic composition, alone or in combination with the second therapeutic agent according to a drug regimen defined according to claim 1 comprising:
(i) prior to treatment with the first therapeutic composition and/or the second therapeutic agent, measuring a first biological sample obtained from the subject for the presence or absence of one or more biomarkers,
(ii) following treatment of the subject with the first therapeutic composition and/or the second therapeutic agent, measuring a second biological sample obtained from the subject for the presence or absence of the one or more biomarkers, and
(iii) comparing the one or more biomarkers measured in the first biological sample with the one or more biomarkers measured in the second biological sample to detect a clinical response, wherein detecting the clinical response corresponds to the presence of a drug effect identifying the subject to be treated.

49. The method of claim 48, wherein the one or more biomarkers comprise the presence of immune cells or cancer cell populations or subpopulations thereof, changes in the expression or activity level of genes and pathways for antigen presentation, or the presence or activity of cross-presenting dendritic cells.

50. The method of claim 48, wherein each respective nanoplexed particle of the first therapeutic composition of the second biological sample is labeled with a fluorophore (BO-11XL).

51. The method of claim 50, further comprising measuring the uptake of the one or more labeled nanoplexed particles into one or more cells present in the second biological sample to identify a drug effect.
